(19) United States
(12) Patent Application Publication
(10) Pub. No.: US 2023/0410993 A1
(43) Pub. Date: Dec. 21, 2023

(54) DEVICES, SYSTEMS, AND METHODS FOR PREDICTING SURGICAL TIME AND OPTIMIZING MEDICAL PROCEDURES AND OUTCOMES

(71) Applicant: MAKO Surgical Corporation, Fort Lauderdale, FL (US)

(72) Inventors: Arman MOTESHAREI, Fort Lauderdale, FL (US); Nathalie WILLEMS, Fort Lauderdale, FL (US); Alison LONG, Fort Lauderdale, FL (US); Daniele DE MASSARI, Fort Lauderdale, FL (US); Hyosig KANG, Weston, FL (US)

(73) Assignee: MAKO Surgical Corporation, Fort Lauderdale, FL (US)

(21) Appl. No.: 18/338,102

(22) Filed: Jun. 20, 2023

Related U.S. Application Data
(60) Provisional application No. 63/353,941, filed on Jun. 21, 2022.

(51) Int. Cl. G16H 40/20; G16H 50/30; G16H 50/70; G16H 20/40; G06T 7/00; G06T 7/62; G06V 20/60; A61B 6/03; A61B 6/00

(52) U.S. Cl. CPC G16H 40/20; G16H 50/30; G16H 50/70; G16H 20/40; G06T 7/0012; G06T 7/62; G06V 20/60; A61B 6/032; A61B 6/54; G06T 2207/30008; G06T 2207/10081; G06V 2201/03

(57) ABSTRACT

Aspects disclosed herein may provide a method for determining a duration of a medical procedure. The method may include receiving imaging data including at least one image acquired of a patient's anatomy, determining at least one parameter of the patient's anatomy based on the imaging data, predicting a duration for the medical procedure based on the determined at least one parameter, and outputting the predicted duration on an electronic display. The at least one parameter may include at least one of a B-score, a joint-space width, an osteophyte position or volume, an alignment, or a deformity based on the imaging data.

### CROSS-REFERENCE TO RELATED APPLICATION

This application claims the benefit of priority under 35 U.S.C. § 119 to U.S. Provisional Patent Application No. 63/353,941, filed Jun. 21, 2022, the entirety of which is incorporated herein by reference.

### FIELD OF THE DISCLOSURE

The present disclosure relates to systems and methods for optimizing medical procedures, and in particular to a system and a method for determining preoperative, intraoperative, and postoperative activities to optimize outcomes after joint replacement procedures.

### BACKGROUND OF THE DISCLOSURE

Musculoskeletal disease presents unique problems for medical practitioners. Surgeries incorporating prosthetics and/or implants such as joint replacement procedures often require careful consideration of various factors, and prolonged surgical times can cause further complications in surgery. Improved systems and methods for performing, collecting, and analyzing data to predict surgical time and outcomes based on surgical time are desired.

### BRIEF SUMMARY OF THE DISCLOSURE

In an aspect of the present disclosure, a method may determine a duration of a medical procedure. The method may include receiving imaging data including at least one image acquired of a patient's anatomy, determining at least one parameter of the patient's anatomy based on the imaging data, predicting a duration for the medical procedure based on the determined at least one parameter, and outputting the predicted duration on an electronic display. The at least one parameter may include at least one of a B-score, a joint-space width, an osteophyte position or volume, an alignment, or a deformity based on the imaging data.

The method may further include identifying at least one femur in the at least one image. The parameter may include a B-score of the identified femur. The method may further include determining that the B-score is greater than a predetermined B-score, and determining that the predicted duration may be longer or shorter than a predetermined duration.

The method may further include identifying at least two bones at a joint in the at least one image. The parameter may include a joint-space width between the at least two bones. The method may include determining whether the joint-space width may be within a predetermined joint-space width range.

The method may further include determining that the joint-space width is outside the predetermined joint-space width range and determining that the predicted duration is longer than a predetermined duration.

The method may further include identifying at least one bone in the at least one image, and detecting at least one osteophyte on the identified at least one bone. The method may further include determining a volume of the detected at least one osteophyte, and determining that the predicted duration may be longer or shorter than a predetermined duration based on the determined volume. Detecting at least one osteophyte on the identified at least one bone may include determining a position of the at least one osteophyte in relation to a predetermined area or compartment on the identified bone.

The method may include identifying at least one bone in the at least one image, determining an alignment parameter of the at least one bone, and determining whether the alignment parameter may be within a predetermined alignment range. The method may include determining that the alignment parameter may be outside the predetermined alignment range, and determining that the predicted duration may be longer than a predetermined duration.

The method may include receiving prior procedure data, the prior procedure data including data from a plurality of prior patients sharing at least one characteristic with the patient. Determining the predicted duration for the medical procedure may be based on the received prior procedure data.

The method may further include receiving at least one of (i) patient specific data regarding the patient, (ii) clinical data relating to the patient, and (iii) surgeon specific data relating to one or more surgeons. Determining the predicted duration for the medical procedure may be based on the received patient specific data, clinical data, and/or surgeon specific data.

The method may further include determining, based on the determined predicted duration for the procedure and/or the at least one parameter of the patient's anatomy, an output.the The output may include at least one of an operating room layout, an operating room schedule, at least one staff member to assist in performance of the medical procedure, a procedure plan, a case difficulty, a risk of infection, a loss of cartilage, a predicted pain perceived by the patient after the procedure, a predicted stress level perceived by the patient after the procedure, a predicted anxiety level perceived by the patient after the procedure, or a predicted mental health status of the patient after the procedure.

The method may further include determining the output may include determining the operating room layout, the operating room schedule, and the at least one staff member. The determined output may be configured to reduce the duration for the procedure.

The method may further include determining, based on the predicted procedure duration, at least one of a case difficulty, a risk of infection, a loss of cartilage, or a predicted pain, stress level, anxiety level, or mental health status of the patient.

The method may further include determining, based on the imaging data, at least one of a bone-to-skin ratio and a bone-to-tissue ratio. Predicting the duration for the medical procedure may be based on the determined bone-to-skin ratio and/or bone-to-tissue ratio.

The method may further include receiving procedure information collected during the medical procedure, and determining a secondary duration for the medical procedure based on the received procedure information.

In another aspect of the present disclosure, a method may determine a duration for a medical procedure. The method may include receiving at least one image acquired of a patient's anatomy, determining, based on the at least one image, a plurality of parameters, predicting a duration for the medical procedure based on the determined plurality of parameters, and outputting the predicted duration on an electronic display. The plurality of parameters may include (i) a B-score, (ii) a joint-space width, (iii) an osteophyte position or volume, and (iv) an alignment or a deformity relating to the patient's anatomy.

Predicting the duration may include determining a longer duration of the medical procedure based on a determined B-score that may be outside a predetermined B-score range, a determined joint-space width that may be outside a predetermined joint-space width range, a determined osteophyte volume that may be outside a predetermined osteophyte volume range, and/or a determined misalignment or severity of the deformity that may be outside of a predetermined alignment range.

In another aspect of the present disclosure, a system may be configured to predict a duration for a medical procedure. The system may include an imaging device configured to acquire at least one image of a patient's anatomy, a memory configured to store information, a controller, and an electronic display. The information may include patient specific information, clinical data, practitioner specific information, preoperative data received from one or more preoperative measurement systems, and prior procedure data related to prior patients that underwent prior procedures. The controller may be configured to execute one or more algorithms to determine, based on the at least one image, at least one parameter of the patient's anatomy, the parameter including at least one of a B-score, a joint-space width, an osteophyte position or volume, an alignment, and a deformity, determine, based on the determined at least one parameter and the stored information in the memory, a duration of the medical procedure to be undergone by a patient, and determine, based on the predicted duration, an output including at least one of an operating room layout, an operating room schedule, at least one staff member to assist in performance of the procedure, a procedure plan, a case difficulty, a risk of infection, a loss of cartilage, or a predicted pain, stress level, anxiety level, or mental health status of the patient after the procedure. The electronic display may be configured to display the determined duration and/or the determined output.

The imaging device may include a computed tomography (CT) imaging device configured to acquire at least one CT scan. The controller may be configured to execute one or more algorithms to determine, based on the at least one CT scan, the osteophyte volume, and determine, based on the determined osteophyte volume, the duration of the medical procedure.

### BRIEF DESCRIPTION OF THE DRAWINGS

A more complete appreciation of the subject matter of this disclosure and the various advantages thereof may be understood by reference to the following detailed description, in which reference is made to the following accompanying drawings:

FIG. 1 is a schematic diagram depicting an electronic data processing system having a procedure time prediction system.

FIG. 2 is a schematic diagram of the electronic data processing system of FIG. 1 depicting interactions among preoperative measurement systems, preoperative data, the procedure time prediction system, outputs, and output systems.

FIG. 3 illustrates a variety of screens or graphical user interfaces that may be displayed on the output systems of FIG. 2.

FIG. 4 depicts an exemplary method of using imaging data to predict procedure time using the electronic data processing system of FIG. 1.

FIG. 5 is a schematic diagram of the electronic data processing system of FIG. 1 depicting interactions among intraoperative measurement systems, intraoperative data, the procedure time prediction system, intraoperatively determined outputs, and output systems.

FIG. 6 depicts an exemplary method of using intraoperative data to update and/or predict procedure time using the electronic data processing system of FIG. 1.

FIG. 7 depicts an exemplary method of using CT scans to predict procedure time based on osteophyte volume using the electronic data processing system of FIG. 1.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

As used herein, the terms “implant trial” and “trial” will be used interchangeably and as such, unless otherwise stated, the explicit use of either term is inclusive of the other term. In this disclosure, “user” is synonymous with “practitioner” and may be any person completing the described action (e.g., surgeon, technician, nurse, etc.).

An implant may be a device that is at least partially implanted in a patient and/or provided inside of a patient's body. For example, an implant may be a sensor, artificial bone, or other medical device coupled to, implanted in, or at least partially implanted in a bone, skin, tissue, organs, etc. A prosthesis or prosthetic may be a device configured to assist or replace a limb, bone, skin, tissue, etc., or portion thereof. Many prostheses are implants, such as a tibial prosthetic component. Some prostheses may be exposed to an exterior of the body and/or may be partially implanted, such as an artificial forearm or leg. Some prostheses may not be considered implants and/or otherwise may be fully exterior to the body, such as a knee brace. Systems and methods disclosed herein may be used in connection with implants, prostheses that are implants, and also prostheses that may not be considered to be “implants” in a strict sense. Therefore, the terms “implant” and “prosthesis” will be used interchangeably and as such, unless otherwise stated, the explicit use of either term is inclusive of the other term. Although the term “implant” is used throughout the disclosure, this term should be inclusive of prostheses which may not necessarily be “implants” in a strict sense.

In describing preferred embodiments of the disclosure, reference will be made to directional nomenclature used in describing the human body. It is noted that this nomenclature is used only for convenience and that it is not intended to be limiting with respect to the scope of the invention. For example, as used herein, the term “distal” means toward the human body and/or away from the operator, and the term “proximal” means away from the human body and/or towards the operator. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such system, process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

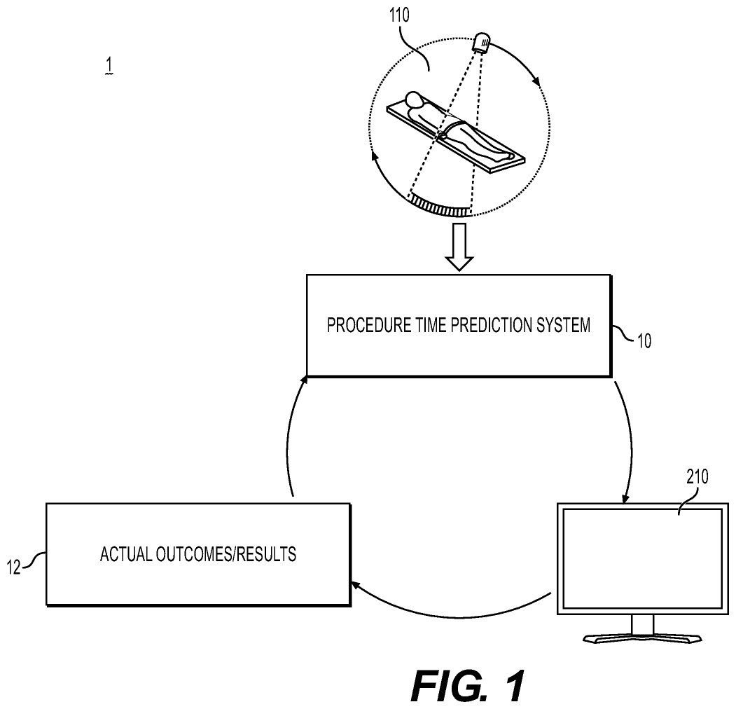

FIG. 1 illustrates an electronic data processing system 1 for collecting, storing, processing, and outputting data during a course of treatment of a patient.

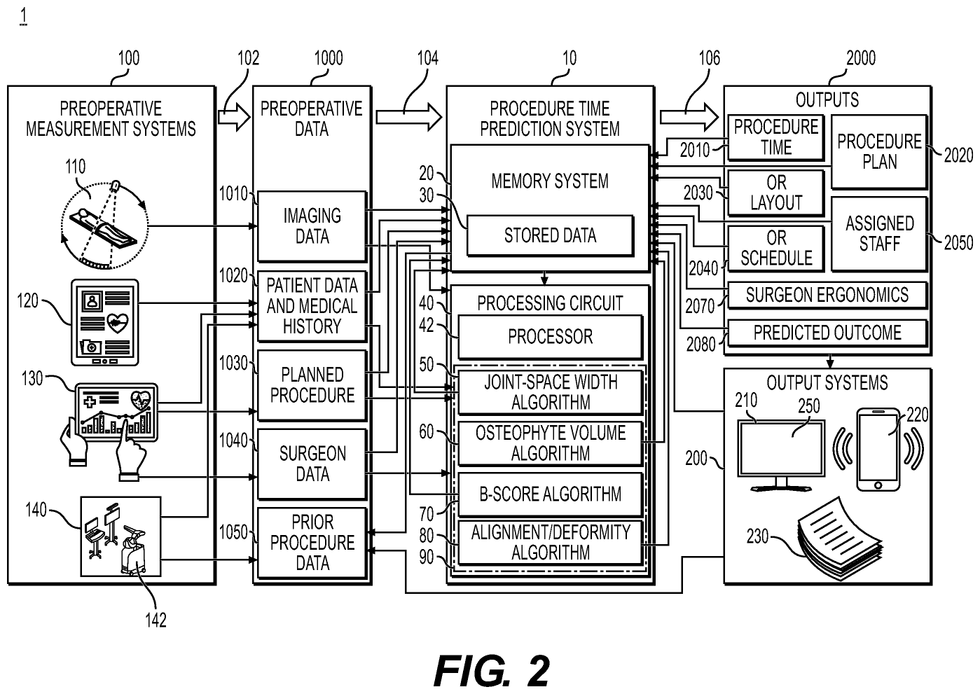

Referring to FIG. 1, the electronic data processing system 1 may include a diagnostic imaging device 110, a procedure time prediction system 10, and an electronic display 210. An instant patient who is planning to undergo a procedure (e.g., surgery) may first undergo imaging using the diagnostic imaging device 110. The procedure time prediction system 10 may analyze images and/or information collected during imaging (which may be transmitted from or stored in the device 110) to predict a time or duration of the planned procedure. The procedure time prediction system 10 may further determine procedure logistics (e.g., procedure scheduling) and/or predicted outcomes (e.g., a risk of complication during the procedure or a risk of infection post-procedure) that are based on the predicted duration. As the course of treatment is continued, actual outcomes and/or results 12 may also be used by the procedure time prediction system 10 to either update its predictions and/or to make future predictions for future patients. The procedure time prediction system 10 may be implemented as one or more computer systems or cloud-based electronic processing systems. Details of the procedure time prediction system 10 are discussed with reference to FIG. 2.

Referring to FIG. 2, the electronic data processing system 1 may include one or more preoperative measurement systems 100 which collect and/or output (via arrow 102) preoperative data 1000 about the instant patient and/or prior patients (e.g., similar prior patients). The procedure time prediction system 10 may receive (via arrow 104) and analyze the preoperative data 1000 and generate one or more outputs or determinations 2000, which may be output (via arrow 106) to one or more output systems 200.

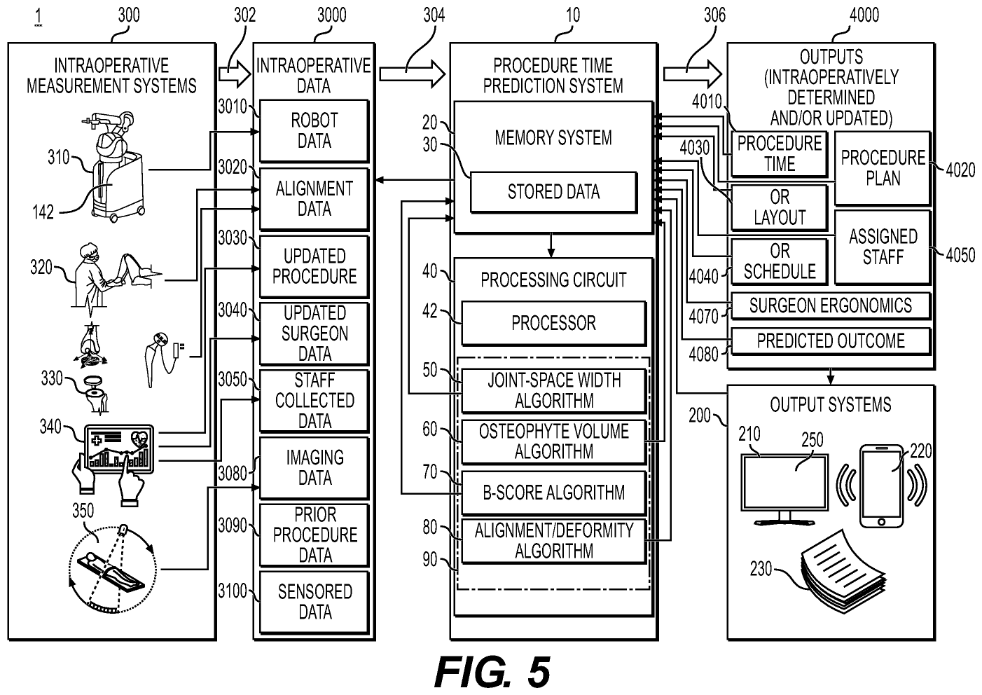

The preoperative measurement systems 100 may include the imaging device 110, electronic devices storing electronic medical records (EMR) 120; patient, practitioner, and/or user interfaces or applications 130 (such as on tablets, computers, or other mobile devices); and a robotic and/or automated data system or platform 140 (e.g., MAKO Robot System or platform, MakoSuite, etc.), which may have a robotic device 142 described in more detail with reference to FIG. 5. The electronic data processing system 1 may collect current imaging data 1010 via the imaging device 110 and supplemental or additional information (e.g., patient data and medical history 1020, planned procedure data 1030, surgeon and/or staff data 1040, and/or prior procedure data 1050) via EMR 120, interfaces 130, sensors and/or electronic medical devices, and/or robotic platform 140. Each of the devices in the preoperative measurement systems 100 (the imaging device 110, EMR 120, user interfaces or applications 130, sensors and/or electronic medical devices, and robotic platform 140) may include one or more communication modules (e.g., WiFi modules, BlueTooth modules, etc.) configured to transmit preoperative data 1000 to each other, to the procedure time prediction system 10, and/or to the one or more output systems 200.

The imaging device 110 may be configured to collect or acquire one or more images, videos, or scans of a patient's internal anatomy, such as bones, ligaments, soft tissues, brain tissue, etc. to provide imaging data 1010, which will be described in more detail later. The imaging device 110 may include a computed tomography (CT) scanner, a magnetic resonance imaging (MM) machine, an x-ray machine, a radiography system, an ultrasound system, a thermography system, a tactile imaging system, an elastography, nuclear medicine functional imaging system, a positron emission tomography (PET) system, a single-photon emission computer tomography (SPECT) system, a camera, etc. The collected images, videos, or scans may be transmitted, automatically or manually, to the procedure time prediction system 10. In some examples, a user may select specific images from a plurality of images taken with an imaging device 110 to be transmitted to the procedure time prediction system 10.

The electronic data processing system 1 may use previously collected data from EMR 120, which may include patient data and medical history 1020 in the form of past practitioner assessments, medical records, past patient reported data, past imaging procedures, treatments, etc. For example, EMR 120 may contain data on demographics, medical history, biometrics, past procedures, general observations about the patient (e.g., mental health), lifestyle information, data from physical therapy, etc. Patient data and medical history 1020 will be described in more detail later.

The electronic data processing system 1 may also collect present or current (e.g., in real time) patient data via patient, practitioner, and/or user interfaces or applications 130. These user interfaces 130 may be implemented on mobile applications and/or patient management websites or interfaces, such as OrthologIQ®. User interfaces 130 may present questionnaires, surveys, or other prompts for practitioners or patients to enter assessments (e.g., throughout a prehabilitation program prior to a procedure), observed psychosocial information and/or readiness for surgery, comments, etc. for additional patient data 1020. Patients may also enter psychosocial information such as perceived or evaluated pain, stress level, anxiety level, feelings, and other patient reported outcome measures (PROMS) into these user interfaces 130. Patients and/or practitioners may report lifestyle information via user interfaces 130. User interfaces 130 may also collect clinical data such as planned procedure 1030 data and planned surgeon and/or staff data 1040 described in more detail later. These user interfaces 130 may be executed on and/or combined with other devices disclosed herein (e.g., with robotic platform 140).

The electronic data processing system 1 may collect prior procedure data 1050 from prior patients and/or other real-time data or observations (e.g., observed patient data 1020) via robotic platform 140. The robotic platform 140 may include one or more robotic devices (e.g., surgical robot 142), computers, databases, etc. used in prior procedures with different patients. The surgical robot 142 may have assisted with, via automated movement, surgeon assisted movement, and/or sensing, a prior procedure and may be implemented as or include one or more automated or robotic surgical tools, robotic surgical or Computerized Numerical Control (CNC) robots, surgical haptic robots, surgical tele-operative robots, surgical hand-held robots, or any other surgical robot. The surgical robot 142 will be described in more detail with reference to FIG.

Although the preoperative measurement system(s) 100 is described in connection with imaging device 1010, EMR 120, user interfaces 130, and robotic platform 140, other devices may be used preoperatively to collect preoperative data 1000. For example, mobile devices such as cell phones and/or smart watches may include various sensors (e.g., gyroscopes, accelerometers, temperature sensors, optical or light sensors, magnetometer, compass, global positioning systems (GPS) etc.) to collect patient data 1020 such as location data, sleep patterns, movement data, heart rate data, lifestyle data, activity data, etc. As another example, wearable sensors, heart rate monitors, motion sensors, external cameras, etc. having various sensors (e.g., cameras, optical light sensors, barometers, GPS, accelerometers, temperature sensors, pressure sensors, magnetometer or compass, MEMS devices, inclinometers, acoustical ranging, etc.) may be used during physical therapy or a prehabilitation program to collect information on patient kinematics, alignment, movement, fitness, heart rate, electrocardiogram data, breathing rate, temperature, oxygenation, sleep patterns, activity frequency and intensity, sweat, perspiration, air circulation, stress, step pressure or push-off power, balance, heel strike, gait, fall risk, frailty, overall function, etc. Other types of systems or devices that may be used in the preoperative measurement system 10 may include electromyography or EMG systems or devices, motion capture (mocap) systems, sensors using machine vision (MV) technology, virtual reality (VR) or augmented reality (AR) systems, etc.

The preoperative data 1000 may be data collected, received, and/or stored prior to an initiation of a medical treatment plan or medical procedure. As shown by the arrows in FIG. 2, the preoperative data 1000 may be collected using the preoperative measurement systems 100, from memory system 20 (e.g., cloud storage system) of the procedure time prediction system 10, and from output systems 200 (e.g., from a prior procedure) for one or more continuous feedback loops. Some of the preoperative data 1000 may be directly sensed via one or more devices (e.g., wearable motion sensors or mobile devices) or may be manually entered by a medical professional, patient, or other party. Other preoperative data 1000 may be determined (e.g., by procedure time prediction system 10) based on directly sensed information, input information, and/or stored information from prior medical procedures.

As previously described, the preoperative data 1000 may include imaging data 1010, patient data and/or medical history 1020, information on a planned procedure 1030, surgeon data 1040, and prior procedure data 1050.

The imaging data 1010 may include morphology and/or anthropometrics (e.g., physical dimensions of internal organs, bones, etc.), fractures, slope or angular data, tibial slope, posterior tibial slope or PTS, bone density, (e.g., bone mineral or bone marrow density, bone softness or hardness, or bone impact), etc. Bone density may be determined separately using the procedure time prediction system 10, as described in more detail later, and/or may be collected or supplemented using, for example, indent tests or a microindentation tool. Imaging data may not be limited to strictly bone data and may be inclusive of other internal imaging data, such as of cartilage, soft tissue, or ligaments.

The imaging data 1010 may be in a form of raw images, videos, or scans collected by the imaging device 110 and to be analyzed by the procedure time prediction system 10. The images or scans may illustrate or indicate bone, cartilage, or soft tissue positions or alignment, composition or density, fractures or tears, bone landmarks (e.g., condyle surface, head or epiphysis, neck or metaphysis, body or diaphysis, articular surface, epicondyle, lateral epicondyle, medial epicondyle, process, protuberance, tubercle vs tuberosity, tibial tubercle, trochanter, spine, linea or line, facet, crests and ridges, foramen and fissure, meatus, fossa and fovea, incisure and sulcus, and sinus), geometry (e.g., diameters, slopes, angles) and/or other anatomical geometry data such as deformities or flare (e.g., coronal plane deformity, sagittal plane deformity, lateral femoral metaphyseal flare, or medial femoral metaphyseal flare). Such geometry is not limited to overall geometry and may include relative dimensions (e.g., lengths or thicknesses of a tibia or femur). The imaging data 1010 may indicate or be used to determine osteophyte size, volume, or positions; bone loss; joint space; B-score; bone quality/density; skin-to-bone ratio; bone loss; hardware detection; anterior-posterior (AP) and medial-lateral (ML) distal femur size, and/or joint angles. Analysis and/or calculations that may be derived from the images or scans will be described in more detail later when describing the procedure time prediction system 10.

In addition to raw images, imaging data 1010 may include intermediate and/or related imaging data 1010 to be used by the procedure prediction system 10 to calculate outputs 2000. Such intermediate imaging data 1010 may include density or composition charts or graphs; quantified data indicating relative positions, dimensions, etc.; and/or processed image data indicating specifically detected attributes, such as a probability of a certain patient condition. One or more algorithms 90 of the procedure prediction system 10 may determine or calculate this intermediate imaging data 1010 in determining outputs 2000, or alternatively or additionally thereto, the imaging device 110 may include one or more processors configured to calculate or quantify, based on the raw images, videos, or scans, at least some of the intermediate imaging data 1010. Intermediate imaging data 1010 may include information relating to, indicating, and/or quantifying aspects of the raw images, charts, etc.

Patient data and medical history 1020 may include information about the instant patient on identity (e.g., name or birthdate), demographics (e.g., patient age, gender, height, weight, nationality, body mass index (BMI), etc.), lifestyle (e.g., smoking habits, exercise habits, drinking habits, eating habits, fitness, activity level, frequency of climbing activities such as up and down stairs, frequency of sit-to-stand movements or bending movements such as when entering and exiting a vehicle, steps per day, activities of daily living or ADLs performed, etc.), medical history (e.g., allergies, disease progressions, addictions, prior medication use, prior drug use, prior infections, frailties, comorbidities, prior surgeries or treatment, prior injuries, prior pregnancies, utilization of orthotics, braces, prosthetics, or other medical devices, etc.), assessments and/or evaluations (e.g., laboratory tests and/or bloodwork, American Society of Anesthesiology or ASA score and/or fitness for surgery or aesthesia) electromyography data (muscle response or electrical activity in response to a nerve's stimulation), psychosocial information (e.g., perceived pain, stress level, anxiety level, mental health status, PROMS (e.g., knee injury and osteoarthritis outcome score or KOOS, hip disability and osteoarthritis outcome score or HOOS, pain virtual analog scale or VAS, PROMIS Global 10 or PROMIS-10, EQ-5D, a mental component summary, satisfaction or expectation information, etc.), past biometrics (e.g., heart rate or heat rate variability, electrocardiogram data, breathing rate, temperature (e.g., internal or skin temperature), fingerprints, DNA, etc.), past kinematics or alignment data, past imaging data, data from prehabilitation programs or physical therapy (e.g., average load bearing time) etc. Medical history 1020 may include prior clinical or hospital visit information, including encounter types, dates of admission, hospital-reported comorbidity data such as Elixhauser and/or Charlson scores or selected comorbidities (e.g., ICD-10 POA), prior anesthesia taken and/or reactions, etc. This list, however, is not exhaustive and preoperative data 1000 may include other patient specific information, clinical information, and/or surgeon or practitioner specific information (e.g., experience level).

Patient data 1020 may come from EMR 120, user interfaces 130, from memory system 20, and/or from robotic platform 140, but aspects disclosed herein are not limited to a collection of the patient data 1020. For example, other types of patient data 1020 or additional data may include data on activity level; kinematics; muscle function or capability; range of motion data; strength measurements and/or force measurements push-off power, force, or acceleration; a power, force, or acceleration at a toe during walking; angular range or axes of joint motion or joint range of motion; flexion or extension data, including step data (e.g., measured by a pedometer), gait data or assessments; fall risk data; balancing data; joint stiffness or laxity data; postural sway data; data from tests conducted in a clinic or remotely; etc.

Information on a planned procedure 1030 may include logistical information about the procedure and substantive information about the procedure. Logistical planned procedure 1030 information may include information about a planned site of the procedure such as a hospital, ambulatory surgery center (ASC), or an operating room; a type of procedure or surgery to be performed (e.g., total or partial knee arthroplasty or replacement, total or partial hip arthroplasty or replacement, spine surgery, patella resurfacing, etc.); scheduling or booking information such as a date or time of the procedure or surgery, planning or setup time, registration time, and/or bone preparation time; a disease or infection state of the surgeon; a name of the primary surgeon or doctor who plans to perform the procedure; equipment or tools required for the procedure; medication or other substances required (e.g., anesthesia type) for the procedure; insurance type or billing information; consent and waiver information; etc. Substantive planned procedure 1030 information may include a surgeon's surgical or other procedure or treatment plan, including planned steps or instructions on incisions, a side of the patient's body to operate on (e.g., left or right) and/or laterality information, bone cuts or resection depths, implant design, type, and/or size, implant alignment, fixation or tool information (e.g., implants, rods, plates, screws, wires, nails, bearings used), cementing versus cementless techniques or implants, final or desired alignment, pose or orientation information (e.g., capture gap values for flexion or extension, gap space or width between two or more bones, joint alignment), planning time, gap balancing time, extended haptic boundary usage, etc. This initial planned procedure 1030 information may be manually prepared or input by a surgeon and/or previously prepared or determined using one or more algorithms.

Surgeon data 1040 may include information about a surgeon or other staff planned to perform the planned procedure 1030. Surgeon data 1040 may include identity (e.g., name), experience level, fitness level, height and/or weight, etc. Surgeon data 1040 may include number of surgeries scheduled for a particular day, number of complicated surgeries scheduled on the day of a planned procedure, average surgery time, etc.

Prior procedure data 1050 may include information about prior procedures performed on a same or prior patient. Such information may include the same type of information as in planned procedure data 1030 (e.g., instructions or steps of a procedure, bone cuts, implant design, implant alignment, etc.) along with outcome and/or result information, which may include both immediate results and long-term results, complications after surgery, length of stay in a hospital, revision surgery data, rehabilitation data, patient motion and/or movement data, etc. Prior procedure data 1050 may include information about prior procedures of prior patients sharing at least one same or similar characteristic (e.g., demographically, biometrically, disease state, etc.) as the instant patient.

Preoperative data 1000 may include any other additional or supplemental information stored in memory system 20, which may also include known data and/or data from third parties, such as data from the Knee Society Clinical Rating System (KSS) or data from the Western Ontario and McMaster Universities Osteoarthritis Index (WOMAC).

The procedure time prediction system 10 may be an artificial intelligence (AI) and/or machine learning system that is “trained” or that may learn and refine patterns between preoperative data 1000, outputs 2000, and actual results 12 (FIG. 1) to make determinations. The procedure time prediction system 10 may be implemented using one or more computing platforms, such as platforms including one or more computer systems and/or electronic cloud processing systems. Examples of one or more computing platforms may include, but are not limited to, smartphones, wearable devices, tablets, laptop computers, desktop computers, Internet of Things (IoT) device, remote server/cloud based computing devices, or other mobile or stationary devices. The procedure time prediction system 10 may also include one or more hosts or servers connected to a networked environment through wireless or wired connections. Remote platforms may be implemented in or function as base stations (which may also be referred to as Node Bs or evolved Node Bs (eNBs)). Remote platforms may also include web servers, mail servers, application servers, etc.

The procedure time prediction system 10 may include one or more communication modules (e.g., WiFi or Bluetooth modules) configured to communicate with preoperative measurement systems 100, output system 200, and/or other third-party devices, etc. For example, such communication modules may include an Ethernet card and/or port for sending and receiving data via an Ethernet-based communications link or network, or a Wi-Fi transceiver for communication via a wireless communications network. Such communication modules may include wired or wireless interfaces (e.g., jacks, antennas, transmitters, receivers, transceivers, wire terminals, etc.) for conducting data communications with external sources via a direct connection or a network connection (e.g., an Internet connection, a LAN, WAN, or WLAN connection, LTE, 4G, 5G, Bluetooth, near field communication (NFC), radio frequency identifier (RFID), ultrawideband (UWB), etc.). Such communication modules may include a radio interface including filters, converters (for example, digital-to-analog converters and the like), mappers, a Fast Fourier Transform (FFT) module, and the like, to generate symbols for a transmission via one or more downlinks and to receive symbols (for example, via an uplink).

The procedure time prediction system 10 may further include the memory system 20 and a processing circuit 40. The memory system 20 may have one or more memories or storages configured to store or maintain the preoperative data 1000, outputs 2000, and stored data 30 from prior patients and/or prior procedures. The preoperative data 1000 and outputs 2000 of an instant procedure may also become stored data 50. Although certain information is described in this specification as being preoperative data 1000 or outputs 2000, due to continuous feedback loops of data (which may be anchored by memory system 20), the preoperative data 1000 described herein may alternatively be determinations or outputs 2000, and the determined outputs 2000 described herein may also be used as inputs into the procedure time prediction system 10. For example, some preoperative data 1000 may be directly sensed or otherwise received, and other preoperative data 1000 may be determined, processed, or output based on other preoperative data 1000. Although the memory system 20 is illustrated close to processing circuit 40, memory system may include memories or storages implemented on separate circuits, housings, devices, and/or computing platforms and in communication with procedure time prediction system 10, such as cloud storage systems and other remote electronic storage systems.

The memory system 20 may include one or more external or internal devices (random access memory or RAM, read only memory or ROM, Flash-memory, hard disk storage or HDD, solid state devices or SSD, static storage such as a magnetic or optical disk, other types of non-transitory machine or computer readable media, etc.) configured to store data and/or computer readable code and/or instructions that completes, executes, or facilitates various processes or instructions described herein. The memory system 20 may include volatile memory or non-volatile memory (e.g., semiconductor-based memory device, a magnetic memory device and system, an optical memory device and system, fixed memory, or removable memory). The memory system 20 may include database components, object code components, script components, or any other type of information structure to support the various activities described herein. In some aspects, the memory system 20 may be communicably connected to the processing circuit 40 and may include computer code to execute one or more processes described herein. The memory system 20 may contain a variety of modules, each capable of storing data and/or computer code related to specific types of functions.

The processing circuit 40 may include a processor 42 configured to execute or perform one or more algorithms 90 based on received data, which may include the preoperative data 1000 and/or any data in the memory system 20 to determine the outputs 2000. The preoperative data 1000 may be received via manual input, retrieved from the memory system 20, and/or received direction from the preoperative measurement systems 100. The processor 42 may be configured to determine patterns based on the received data.

The processor 42 may be implemented as a general purpose processor or computer, special purpose computer or processor, microprocessor, digital signal processor (DSPs), an application specific integrated circuit (ASIC), one or more field programmable gate arrays (FPGAs), a group of processing components, processor based on a multi-core processor architecture, or other suitable electronic processing components. The processor 42 may be configured to perform machine readable instructions, which may include one or more modules implemented as one or more functional logic, hardware logic, electronic circuitry, software modules, etc. In some cases, the processor 42 may be remote from one or more of the computing platforms comprising the procedure time prediction system 10. The processor 42 may be configured to perform one or more functions associated with the procedure time prediction system 10, such as precoding of antenna gain/phase parameters, encoding and decoding of individual bits forming a communication message, formatting of information, and overall control of one or more computing platforms comprising the procedure time prediction system 10, including processes related to management of communication resources and/or communication modules.

In some aspects, the processing circuit 50 and/or memory system 20 may contain several modules related to medical procedures, such as an input module, an analysis module, and an output module. The procedure time prediction system 10 need not be contained in a single housing. Rather, components of the procedure time prediction system 10 may be located in various different locations or even in a remote location. Components of the procedure time prediction system 10, including components of the processing circuit 40 and the memory system 20, may be located, for example, in components of different computers, robotic systems, devices, etc. used in surgical procedures.

The procedure time prediction system 10 may use the one or more algorithms 90 to make intermediate determinations and to determine the one or more outputs 2000. The one or more algorithms 90 may be configured to determine or glean data from the preoperative data 1000, including the imaging data 1010. For example, the one or more algorithms 90 may be configured for bone recognition, soft tissue recognition, and/or to make determinations related to the intermediate imaging data 1010 previously described.

The one or more algorithms 90 may be machine learning algorithms that are trained using, for example, linear regression, random forest regression, CatBoost regression, etc. The one or more algorithms 90 may be continuously modified and/or refined based on actual outcomes and/or results 12 (FIG. 1). The one or more algorithms 90 may be configured to use segmenting techniques and/or thresholding techniques on received images, videos, and/or scans of the imaging data 1010 to determine the previously described intermediate imaging data 1010 and/or the one or more outputs 2000. For example, the one or more algorithms 90 may be configured to segment an image (e.g., a CT scan), threshold soft tissue, generate a .txt comparisons of certain identified bones or tissues (e.g., tibia and femur), and run code to extract values (e.g., PPT or PTT) and populate a database. The one or more algorithms 90 may be configured to automate data extraction and/or collection upon receiving an image from the imaging device 110.

As will be described in more detail in connection with the one or more algorithms 90 and the output system 200, the one or more outputs 2000 may include a predicted procedure time or duration 2010, a procedure plan 2020, an operating room layout 2030, an operating room schedule 2040, assigned or designated staff 2050, recommended surgeon ergonomics 2070, and predicted outcomes 2080 of the procedure. The predicted procedure time 2010 may be a total time or duration of a procedure (e.g., as outlined in the procedure plan 2020), and may further include a time or duration of small steps or processes of the procedure. In some examples, the predicted procedure time 2010 may be a predicted time to complete a portion of a procedure. The procedure plan 2020, the operating room layout 2030, the operating room schedule 2040, the assigned staff 2050, the recommended surgeon ergonomics 2070, and the predicted outcomes 2080 may be determined based on the determined predicted procedure time 2010. The predicted outcomes 2080 may include a predicted perceived pain level for the patient, a predicted stress level, anxiety level, and/or mental health status of the patient, a predicted cartilage loss, a predicted risk of infection, a rating of a case difficulty, etc. The predicted outcomes 2080 may also include predictions and/or risks if, during the procedure, a time exceeds (or alternatively, is less than) the predicted procedure time 2010 (for example, how a risk of complication and/or a risk of infection may increase based on the procedure taking longer than the predicted procedure time 2010).

The one or more algorithms 90 may include a joint-space width algorithm 50, an osteophyte volume algorithm 60, a B-score algorithm 70, and an alignment/deformity algorithm 80. Alternatively, one or more of these algorithms may be combined. For example, the joint-space width algorithm 50, the osteophyte volume algorithm 60, the B-score algorithm 70, and the alignment/deformity algorithm 80 may be combined in a single or master algorithm. Each of the joint-space width algorithm 50, the osteophyte volume algorithm 60, the B-score algorithm 70, and the alignment/deformity algorithm 80 may be configured to use not only preoperative data 1000 as input but also determinations and/or outputs 2000 from each other. Each of the one or more algorithms 90 (the joint-space width algorithm 50, the osteophyte volume algorithm 60, the B-score algorithm 70, and the alignment/deformity algorithm 80) may be configured to use image processing techniques to recognize or detect bones, tissues, bone landmarks, etc. and calculate or predict dimensions and/or positions thereof. The one or more algorithms are not limited to determinations relating to joint-space width, osteophyte volume, B-score, and alignment/deformity, and may include and/or be configured to make other procedural determinations, such as those relating to joint laxity or stiffness, discharge time or length of stay time, frailty, fall risk, balancing assessments, patient readiness, etc.

A joint space width (JSW) may be a distance between two or more bones at a joint. The joint-space width algorithm 50 may be configured to determine one or more JSW parameters from the preoperative data 1000 (e.g., imaging data 1010) relating to a joint space width in one or more target joints. The one or more JSW parameters may include joint space widths at predetermined locations, joint space widths across different directions (e.g., medial JSW or lateral JSW), average or mean joint space width (e.g., mean three-dimensional or 3D joint space width), changing joint-space (e.g., joint space narrowing), an average or mean joint space narrowing (e.g., mean 3D joint space narrowing), impingement data, impingement angles, impingement data based on a predicted or determined implant, etc. The joint-space width algorithm 50 may detect and/or reference a plurality (e.g., hundreds) of bone landmarks to determine joint space widths at various positions.

The joint-space width algorithm 50 may assess one or more of these JSW parameters at various anatomical compartments (e.g., anterior lateral, anterior medial, central lateral, central medial, posterior lateral, posterior medial) of one or more bones (e.g., tibia and femur). The joint space width algorithm 50 may also be configured to predict joint spaces based on loadbearing and/or unloaded conditions using other preoperative data 1000, such as kinematics data or activity level data.

The joint space width algorithm 50 may, based on supplemental patient data 1030, determine whether a joint space width is decreasing or narrowing (and/or increasing or widening) based on a comparison of previously measured joint space widths and/or based on a comparison of imaging data from previous image acquisitions. The joint space width algorithm 50 may also determine cartilage thickness or determine predict a cartilage loss during the procedure (e.g., by using a Z-score or other statistical measure).

For example, The joint-space width algorithm 50 may also be used to determine scores or values in a plurality (e.g., four) of anatomical compartments (e.g., knee joint) based on joint-space width or cartilage loss, and determine a composite score or C-score based on the determined scores of each of the compartments. The scores for each compartment and/or the C-score may also be based on patient data 1020, such as gender, as males and females on average have different cartilage widths. The joint-space width algorithm 50 may determine or select a compartment among the plurality of compartments that should be resurfaced during the procedure, and determine that the procedure plan 2020 should include one or more steps directed to resurfacing the selected compartment. The joint-space width algorithm 50 may determine cartilage thickness or loss based on a determined C-score, and may consider patient data 1020 (e.g., gender). The joint-space width algorithm 50 may convert a joint-space width (e.g., in mm) to a Z-score or other score. A Z-score may describe a relationship between a particular value (e.g., joint-space width) with a mean or average of a group of values. For example, a Z-score may be measured in terms of standard deviations from the mean such that Z-score of 0 may indicate a value that is identical to the mean score. In some examples, the joint-space width algorithm 50 may determine patient data 1020, such as gender, based on the determined JSW parameters (e.g., C-score or Z-score). In some examples, the joint-space width algorithm 50 may determine whether the procedure plan 2020 should include a total or partial arthroplasty (e.g., a total or partial knee arthroplasty).

Based on the determined JSW parameters, the joint-space width algorithm 50 and/or the one or more algorithms 90 collectively may be used to determine one or more of the outputs 2000. In some examples, the joint-space width algorithm 50 may determine and/or predict (or be used to determine and/or predict) a procedure time or duration 2010 to execute a procedure plan 2020. For example, the joint-space width algorithm 50 may determine that a joint space width of a patient is outside of a predetermined range, is narrowing over time and/or is smaller than a first predetermined threshold, or is widening over time and/or is greater than a second predetermined threshold. The procedure time prediction system 10 may, based at least in part on these determinations by the JSW algorithm 50, predict a longer or shorter procedure time 2010 (for example, based on a function where the predicted time is inversely proportional or proportional to the joint space width, and/or based on a step-wise increase based on predetermined thresholds, etc.) Other factors (e.g., from patient data 1020) may change the analysis and/or relationship such that the procedure time prediction system 10 and/or the osteophyte joint-space width algorithm 50 may determine certain relationships between higher or lower JSW parameters combined with certain patient data 1020. Details of the other outputs 2000 will be described in more detail hereinafter in connection with all of the algorithms 90.

An osteophyte may be a bone spur that develops on a bone. Osteophyte volume may refer to a total volume of osteophytes on a bone or a specific portion of a bone. The osteophyte volume algorithm 60 may be configured to detect or recognize one or more osteophytes at a target bone, joint, or portion of a bone, and determine or calculate one or more osteophyte parameters from the preoperative data 1000 (e.g., imaging data 1010) relating to osteophyte detection or osteophyte dimensions (e.g., volume) of one or more detected osteophytes in one or more target joints). The one or more osteophyte parameters may include an osteophyte location, an osteophyte number, osteophyte volumes at predetermined locations, osteophyte areas across different directions (e.g., medial or lateral), an average or mean osteophyte volume, changing or progressing osteophyte volume, impingement data, impingement angles, impingement data based on a predicted or determined implant, etc. The osteophyte volume algorithm 60 may assess one or more of these osteophyte parameters at one or more bones (e.g., femur or tibia) and/or various anatomical compartments (e.g., anterior lateral, anterior medial, central lateral, central medial, posterior lateral, posterior medial) of one or more bones (e.g., tibia and femur). The osteophyte volume algorithm 60 may also be configured to predict osteophyte volume or progression based on other preoperative data 1000, such as kinematics data or activity level data.

The osteophyte volume algorithm 60 may, based on supplemental patient data 1030, determine whether osteophyte volume (e.g., total osteophyte volume or an osteophyte volume of a specific region or osteophyte) is increasing or decreasing based on a comparison of previously measured osteophyte volumes and/or based on a comparison of imaging data from previous image acquisitions. The osteophyte volume algorithm 60 may further determine, predict, or diagnose a disease state or a disease progression (e.g., osteoarthritis or OA) based on the determined osteophyte parameters.

Based on the determined osteophyte parameters, the osteophyte volume algorithm 60 and/or the one or more algorithms 90 collectively may be used to determine one or more of the outputs 2000. The osteophyte volume algorithm 60 may determine and/or predict (or be used to determine and/or predict) the procedure time 2010 to execute the procedure plan 2020. For example, the osteophyte volume algorithm 60 may determine that an osteophyte volume of a patient is progressing over time and/or is larger than a predetermined threshold, and predict a longer procedure time 2010 (for example, based on a function where the predicted time is proportional to the osteophyte volume, and/or based on a step-wise increase based on predetermined thresholds, etc.) However, a higher osteophyte volume may not necessarily result in a longer procedure time 2010, as other factors (e.g., from patient data 1020) may change the analysis such that the procedure time prediction system 10 and/or the osteophyte volume algorithm 60 may determine different relationships between higher or lower osteophyte volumes combined with certain patient data 1020 (e.g., shorter procedure time 2010 based on a higher osteophyte volume and certain patient data 1020). Details of the other outputs 2000 will be described in more detail hereinafter.

B-score may be a type of score or scoring system based on and/or quantifying a shape of a femur or knee joint. B-score may be a holistic, average, or overall score indicating an overall assessment of the femur and/or the knee, but knees having different specific complications or deformities may result in similar B-scores. The B-score may be based on how the shape of the femur compares to knee shapes of those with OA and knee shapes of those who do not have OA, and may be determined using, for example, statistical shape modelling (SSM) or other processes. B-score may be a continuous, quantitative variable, which may be used to quantify overall amount of OA damage in the knee, and also to measure progression in longitudinal studies.

As OA progresses, each bone may exhibit a characteristic shape change, involving osteophyte growth around cartilage plates, and a spreading and flattening of a subchondral bone. A femur shape change may increase regardless of an anatomical compartment affected, and may be more sensitive to change than the tibia and patella. The B-score may represent a distance along the “OA” shape change in the femur bone.

In some examples, a B-score may be recorded as a z-score, similar to a T-score in osteoporosis, which may represent units of standard deviation (SD) of a healthy population, with 0 defined as ae mean of a healthy population. Values of −2 to +2 may represent a healthy population, whereas values above+2 may fall beyond the healthy population.

The B-score algorithm 70 may be configured to determine a B-score from imaging data 1080 containing images and/or related data of a knee and/or femur. The B-score may be based in part on, or correlate to, OA progression, where a B-score of 0 may correlate to and/or indicate a mean femur shape of those who do not have OA. Further details of how B-score is calculated may be found in “Machine-learning, MM bone shape and important clinical outcomes in osteoarthritis: data from the Osteoarthritis Initiative” by Michael A. Bowes, Katherine Kacena, Oras A. Alabas, Alan D. Brett, Bright Dube, Neil Bodick, Philip G Conaghan published Nov. 13, 2020, which is incorporated reference herein in its entity. Aspects disclosed herein are not limited to such a B-score, however. For example, the B-Score algorithm 70 may additionally and/or alternatively calculate other scores or quantifications of other bone shapes based on how they compare to bone shapes of those having a particular disease.

The B-score algorithm 70 may be configured to detect or recognize one or more target bones or joint (e.g., femur), detect or recognize a shape of the target bone or joint, and/or determine or calculate one or more shape score parameters from the preoperative data 1000 (e.g., imaging data 1010) relating to the shape of the target bone and/or how that shape compares with prior patients having a particular disease. For ease of description, an example where the B-score algorithm 70 calculates one or more B-score parameters in connection to knee and/or femur will be described. The one or more B-score parameters may include B-scores at different times or in different images, an average or mean B-score, and/or a changing or progressing B-score. The B-score algorithm 70 may also be configured to predict a future B-score or B-score progression based on other preoperative data 1000, such as kinematics data or activity level data.

The B-score algorithm 70 may, based on supplemental patient data 1030, determine whether a B-score for a particular femur (e.g., left femur) or both femurs is increasing or decreasing based on a comparison of previously measured B-scores and/or based on a comparison of imaging data from previous image acquisitions. The B-score algorithm 70 may further determine, predict, or diagnose a disease state or a disease progression (e.g., osteoarthritis or OA) based on the determined B-score and/or B-score progression.

Based on the determined B-score, the B-score algorithm 70 and/or the one or more algorithms 90 collectively may be used to determine one or more of the outputs 2000. The B-score algorithm 70 may determine and/or predict (or be used to determine and/or predict) the procedure time 2010 to execute the procedure plan 2020. For example, the B-score algorithm 70 may determine that certain patient data 1020 combined with a B-score of a patient is progressing over time and/or is larger than a predetermined threshold, and predict a longer procedure time 2010 (for example, based on a function where the predicted time is proportional to the B-score, and/or based on a step-wise increase based on predetermined thresholds, etc.)

However, a higher B-score may not necessarily result in a longer procedure time 2010. For example, patients belonging to the U.S. population that have a higher B-score may be associated with longer procedure times, while patients belonging to EU populations that have a higher B-score may be associated with shorter procedure times. Thus, the B-score algorithm 70 and/or procedure time prediction system 10 may determine a longer procedure time 2010 based on a higher B-score and a patient nationality of U.S. and a shorter procedure time 2010 based on a higher B-score and a patient nationality of an EU country. Other factors (e.g., from patient data 1020) may change the analysis and/or relationship such that the procedure time prediction system 10 and/or the B-score algorithm 70 may determine certain relationships between higher or lower B-scores combined with certain patient data 1020. Details of the other outputs 2000 will be described in more detail hereinafter.

Alignment and/or deformity may refer to how two or more bones are positioned and/or moved as compared to a healthy patient having a healthy alignment at the two or more bones. The alignment/deformity algorithm 80 may be configured to detect or recognize one or more target bones or joints, detect relative positions and/or dimensions of the one or more target bones or joints, and determine or calculate one or more alignment/deformity parameters from the preoperative data 1000 (e.g., imaging data 1010) relating to alignment detection or osteophyte dimensions (e.g., volume) of one or more detected osteophytes in one or more target joints).

The one or more alignment/deformity parameters may include alignment and/or relative position data at certain locations (e.g., joint location), across different directions (e.g., medial or lateral), an average or mean alignment and/or an alignment score, changing or progressing alignment, alignment based on a predicted or determined implant, etc. The alignment/deformity algorithm 80 may assess one or more of these alignment/deformity parameters at one or more bones (e.g., femur or tibia) and/or various anatomical compartments (e.g., anterior lateral, anterior medial, central lateral, central medial, posterior lateral, posterior medial) of one or more bones (e.g., tibia and femur). The alignment/deformity algorithm 80 may also be configured to predict alignment or progression based on other preoperative data 1000, such as kinematics data or activity level data.

The one or more alignment/deformity parameters may include alignment and/or relative positions (e.g., relative to anatomical and/or mechanical axes), such as lower extremity mechanical alignment, lower extremity anatomical alignment, femoral articular surface angle, tibial articular surface angle, mechanical axis alignment strategy, anatomical alignment strategy, natural knee alignment strategy, femoral bowing, varus-valgus deformity and/or angles, tibial bowing, patello-femoral alignment, coronal plane deformity, sagittal plane deformity, extension motion, flexion motion, anterior cruciate ligament (ACL) ligament intact, posterior cruciate ligament (PCL) ligament intact, knee motion and/or range of motion data (e.g., collected with markers appearing in the raw images, videos, or scans) in all three planes during active and passive range of motion in a joint, three dimensional size, quantified data indicating proportions and relationships ofjoint anatomy in both static and motion, quantified data indicating height of a joint line, metaphyseal flare, medial femoral metaphyseal flare, proximal tibio-fibular joint, coronal tibial diameter, femoral interepicondylar diameter, femoral intermetaphyseal diameter, sagittal tibial diameter, posterior femoral condylar offset-medial and lateral, lateral epicondyle to joint line distance, and/or tibial tubercle to joint line distance. However, aspects disclosed herein are not limited to these alignment parameters.

The one or more alignment/deformity parameters may include data on bone landmarks (e.g., condyle surface, head or epiphysis, neck or metaphysis, body or diaphysis, articular surface, epicondyle, process, protuberance, tubercle vs tuberosity, trochanter, spine, linea or line, facet, crests and ridges, foramen and fissure, meatus, fossa and fovea, incisure and sulcus, and sinus) and/or bone geometry (e.g., diameters, slopes, angles) and other anatomical geometry data. Such geometry is not limited to overall geometry and may include specific lengths or thicknesses (e.g., lengths or thicknesses of a tibia or femur). Imaging data 1010 may also include data on soft tissues for ligament insertions and/or be used to determine ligament insertion sites.

The alignment/deformity algorithm 80 may, based on imaging data 1080 and/or supplemental patient data 1020, determine whether a misalignment, deformity, distances between certain bones, and/or angles between different bones is increasing or decreasing based on a comparison of previously measured alignment/deformity parameters and/or based on a comparison of imaging data from previous image acquisitions. The alignment/deformity algorithm 80 may further determine, predict, or diagnose a disease state or a disease progression (e.g., osteoarthritis or OA) based on the determined alignment/deformity parameters.

Based on the determined alignment/deformity parameters, the alignment/deformity algorithm 80 and/or the one or more algorithms 90 collectively may be used to determine one or more of the outputs 2000. The alignment/deformity algorithm 80 may determine and/or predict (or be used to determine and/or predict) the procedure time 2010 to execute the procedure plan 2020. For example, the alignment/deformity algorithm 80 may determine that a deformity of a patient is progressing over time and/or is larger than a predetermined threshold, and predict a longer procedure time 2010 (for example, based on a function where the predicted time is proportional to the osteophyte volume, and/or based on a step-wise increase based on predetermined thresholds, etc.) Other factors (e.g., from patient data 1020) may change the analysis and/or relationship such that the procedure time prediction system 10 and/or the alignment/deformity algorithm 80 may determine certain relationships between higher or lower alignment/deformity parameters combined with certain patient data 1020. For example, although alignment/deformity algorithm 80 may determine that a deformity of a patient is minor and/or improving, the alignment/deformity algorithm 80 and/or the procedure time prediction system 10 may determine a longer procedure time 2010 based on a location of the deformity and/or other patient data 1020 (e.g., gender, height, etc.) Details of the other outputs 2000 will be described in more detail hereinafter.

The one or more algorithms 90 may operate simultaneously (or alternatively, at different times throughout the preoperative and intraoperative periods) and exchange inputs and outputs. The one or more algorithms 90 may be configured to determine other scores, values, and/or parameters and are not limited to joint space width, osteophyte volume, B-score, alignment/deformity, and/or a patient readiness score. For example, the one or more algorithms 90 may be configured to determine scores related to bone density (e.g., T-score), joint stiffness or laxity, patient readiness, bone-to-skin ratio, etc. A patient readiness score may preoperatively indicate a patient's independence and/or readiness to undergo a procedure (e.g., surgery) or if further prehabilitation may be needed to enhance a recovery time post-operatively. In addition, the patient readiness score may intraoperatively or postoperatively indicate a patient's readiness to be discharged from a hospital after the procedure. The procedure time prediction system 10 may be configured to determine a time period (e.g., number of days) for the patient to wait for the procedure and/or determine other scheduling parameters for the procedure.

The one or more algorithms 90 may be configured for bone recognition and may also be configured to detect or determine prepatellar thickness (PPT) and/or pretubercular thickness (PTT), a minimum distance from bone to skin, tissue-to-bone ratio, bone-to-tissue distances or values, and/or bone-to-tissue distances for PPT and/or PTT, bone-to-skin ratio, etc.

The procedure time prediction system 10 may determine, from the parameters determined from the one or more algorithms 90, the procedure time 2010. For example, the procedure time prediction system 10 may determine a longer procedure time 2010 based on a narrower (or narrowing) joint space width and/or a wider (or widening) joint space width determined by the joint space width algorithm 50, a larger (or increasing) osteophyte number and/or volume determined by the osteophyte volume algorithm 60, a larger (or increasing) B-score determined by the B-score algorithm 70, a larger (or increasing) deformity and/or misalignment determined by the alignment/deformity algorithm 80, and/or a larger bone-to-tissue ratio, PPT, and/or PTT determined by the one or more algorithms 90, along with certain combinations of patient data 1020. However, these factors are an example of what may yield a longer procedure time 2010, and may be changed based on other factors of combinations based on other inputs 1000, such as those from patient data 1020. The procedure time prediction system 10 may be configured to determine new relationships based on certain combinations to more accurately determine procedure time 2010. For example, the procedure time prediction system 10 system may determine over time that although a higher B-score in US patients may result in a longer procedure time 2010, a higher B-score in EU patients may result in a shorter procedure time 2010. Other combinations and/or factors may further change the analysis and/or relationships of all inputs 1000, parameters determined from the algorithms 90, and the outputs 2000 (e.g., procedure time 2010).

PPT and/or PTT may be a distance measurement between a bone and skin determined using images (e.g., CT scans), and may be used as a proxy or alternative to a manually input BMI. In some examples, PPT and/or PTT at a joint (e.g., knee joint) may provide more precise information than BMI, which may be a whole-body measurement. The procedure time prediction system 10 may determine a longer procedure time 2010 based on a larger bone-to-tissue ratio, PPT, and/or PTT determined by the one or more algorithms 90 and/or a larger BMI (e.g., input and/or determined by the one or more algorithms 90), as practitioners may need more time to handle (e.g., cut through) a larger amount of tissue. In addition, the procedure time prediction system 10 may determine a higher case difficulty level based on a larger bone-to-tissue ratio, PPT, and/or PTT determined by the one or more algorithms 90, as a joint (e.g., knee) may be harder to balance due to more tissue.

As an example in the context of a knee surgery, the joint-space width algorithm 50 may determine that a medial space width is narrowing over time and/or is smaller than a predetermined threshold, the osteophyte volume algorithm 60 may determine that an osteophyte volume in the femur is increasing over time, the B-score algorithm 70 may determine that a B-score of the femur is larger (e.g., 3 or greater) than an average B-score for a similarly situated patient, and the alignment/deformity algorithm 80 may determine that the patient has a varus-valgus deformity, and the procedure time prediction system 10 may predict a longer procedure time 2010 for a total knee arthroplasty.

The one or more algorithms 90 may also determine (or be used by the procedure time prediction system 10) to determine other aspects of the procedure plan 2020, such as steps, instructions, tools, etc. for preparing for and/or performing a procedure (e.g., surgery). The procedure plan 2020 may include a planned number, position, length, slope, angle, orientation, etc. of one or more tissue incisions or bone cuts, a planned type of the implant, a planned design (e.g., shape and material) of the implant, a planned or target position or alignment of the implant, a planned or target fit or tightness of the implant (e.g., based on gaps and/or ligament balance), a desired outcome (e.g., alignment of joints or bones, bone slopes such as tibial slopes, activity levels, or desired values for postoperative outputs 2000), a list of steps for the surgeon to perform, a list of tools that may be used, etc. The procedure time prediction system 10 may determine, based on a longer predicted procedure duration 2010, that a type or extent of the procedure in the procedure plan 2020 should include a more corrective surgery, such as from a partial joint (e.g., knee, hip, or shoulder) replacement to a total joint replacement, that certain fixation or other techniques should be used, whether cementing techniques or cementless techniques or implants should be used, etc.

The procedure plan 2020 may, for example, include instructions on how to prepare a proximal end of a tibia to receive a tibial implant, how to prepare a distal end of a femur to receive a femoral implant, how to prepare a glenoid or humerus to receive a glenoid sphere and/or humeral prosthetic component, how to prepare a socket area or acetabulum to receive a ball joint, etc. The bone surface may be cut, drilled, or shaved relative to a reference (e.g., a transepicondylar axis). The procedure plan 2020 may include positions, lengths, and other dimensions for the surfaces and/or values for the slopes for bone preparation. As will be described later, the procedure plan 2020 may be updated and/or modified based on intraoperative data 3000.

The procedure plan 2020 may also include predictive or target outcomes and/or parameters, such as target postoperative range of motion and alignment parameters, and target scores (e.g., stability, fall risk, joint stiffness or laxity, or OA progression). These target parameters may ultimately be compared postoperatively to corresponding measured postoperative data or results to determine whether an optimized outcome for a patient was achieved. The procedure time prediction system 10 may be configured to update the procedure plan 2020 based on manual input and/or feedback input by practitioners, newly acquired preoperative data 1000, or patient feedback.

The procedure time prediction system 10 may determine, based on a joint-space width determined by the joint-space width algorithm 50 and/or alignment/deformity parameters determined by the alignment/deformity algorithm 70, that the procedure plan 2020 should include a certain implant design or dimensions. For example, based on a determined joint-space width or joint-space narrowing by the joint-space width algorithm 50, the procedure time prediction system 10 may determine that an implant width should be decreased and/or determine a type of implant (e.g., a constrained type) based on a narrower determined joint-space width or joint-space narrowing. Based on a joint width and/or an increased joint-space width determined by the joint-space width algorithm 50 and/or a looser or less stable joint determined by alignment/deformity algorithm 70, the procedure time prediction system 10 may determine that an implant width should be increased (e.g., with augments or shims) and/or determine a type of implant should be a stabilizing or constrained type of implant, that a type or extent of procedure in the procedure plan 2020 should include a more corrective surgery, such as from a partial joint (e.g., knee, hip, or shoulder) replacement to a total joint replacement, etc.

The procedure time prediction system 10 may determine (or be used to determine) that the predicted outcomes 2080 may include a certain perceived pain level, a predicted stress level, anxiety level, and/or mental health status of the patient, a certain recovery time, certain risks of infection, certain risks of complications during a procedure (e.g., breathing difficulties and/or blood flow or heart rate complications), certain risks or likelihood of revision surgery, and a rating of difficulty for a case. With respect to cartilage loss, the procedure time prediction system 10 may determine a Z score or other statistical measure to determine a risk of cartilage loss. The determined predicted cartilage loss may be based on the joint space width.

For example, the procedure time prediction system 10 may predict an increased perceived pain level, a predicted stress level, anxiety level, and/or mental health status of the patient, an increased recovery time, an increased risk of complications during the procedure, an increased risk of infection, an increased likelihood of revision surgery, and/or an increased difficulty rating based on a comparison of the determined joint space width by the joint space width algorithm 50 with a planned implant size in the determined procedure plan 2020, based on a narrower joint space width determined by the joint space width algorithm 50, based on joint space narrowing over time determined by the joint space width algorithm 50, based on a larger osteophyte volume or osteophyte number determined by the osteophyte volume algorithm 60, based on an increasing or progressing osteophyte volume determined by the osteophyte volume algorithm 60, based on a higher or increasing B-score (or alternatively, a B-score outside of a predetermined range) determined by the B-score algorithm 70, based on a severe deformity detected by the alignment/deformity algorithm 80, based on an OA progression determined using the one or more algorithms 90, based on impingement data calculated using parameters determined from the joint space width algorithm 50, the osteophyte volume algorithm 60, and/or the alignment/deformity algorithm 80, a larger bone-to-tissue ratio, PPT, and/or PTT, etc.

Similarly, the procedure time prediction system 10 may predict a decrease perceived pain level, a decreased stress level or anxiety level of the patient, and increased mental health status of the patient, a decreased recovery time, a decreased risk of complication during the procedure, a decreased risk of infection, a decreased likelihood of revision surgery, and/or a decreased difficulty rating based on a comparison of the determined joint space width by the joint space width algorithm 50 with a planned implant size in the determined procedure plan 2020, based on a joint space width within a predetermined range determined by the joint space width algorithm 50, based on a slower joint space narrowing or widening over time and/or a joint space remaining constant over time determined by the joint space width algorithm 50, based on a lower osteophyte volume or osteophyte number determined by the osteophyte volume algorithm 60, based on a slower progressing and/or constant osteophyte volume determined by the osteophyte volume algorithm 60, based on a lower and/or constant B-score determined by the B-score algorithm 70, based on a healthier alignment and/or a less severe deformity detected by the alignment/deformity algorithm 80, based on a lower OA progression determined using the one or more algorithms 90, based on impingement data calculated using parameters determined from the joint space width algorithm 50, the osteophyte volume algorithm 60, and/or the alignment/deformity algorithm 80, a smaller bone-to-tissue ratio, PPT, and/or PTT, etc.

The procedure time prediction system 10 may also determine, assign, and/or designate assigned staff 2020 to assist in performance of the procedure. For example, the procedure time prediction system 10 may determine that the assigned staff 2020 should include surgeons, nurses, or other individuals having more experience with a type of surgery (e.g., knee surgery or total knee arthroplasty) planned in the procedure plan 2020 and/or having more experience with patients having similar characteristics as the instant patient (e.g., narrower joint space width, patient history, a certain type of deformity etc.) The procedure time prediction system 10 may determine that the assigned staff 2020 should include surgeons, nurses, or other individuals having experience with procedures that take as long as the predicted procedure time 2010. The procedure time prediction system 10 may store or determine experience scores or levels for each staff member, and may determine an average of a composite procedure or staff team and/or use a rolling average to determine the assigned staff 2020.

The procedure time prediction system 10 may determine that the assigned staff 2020 should have, individually and/or collectively, more experience based on: a certain type or more complex implant plan, a narrower (or narrowing over time) joint space width determined by the joint space width algorithm 50, a larger osteophyte volume or osteophyte number (or increasing osteophyte volume or number over time, or an osteophyte volume outside of a predetermined range) determined by the osteophyte volume algorithm 60, a higher (or increasing) B-score determined by the B-score algorithm 70, a severe or complicated deformity detected by the alignment/deformity algorithm 80, an OA progression determined using the one or more algorithms 90, impingement data calculated using parameters determined from the joint space width algorithm 50, the osteophyte volume algorithm 60, and/or the alignment/deformity algorithm 80, etc.

The procedure time prediction system 10 may also determine an operating room layout 2030 and an operating room schedule 2040 based on joint-space width parameters determined by the joint-space width algorithm 50, osteophyte volume parameters determined by the osteophyte volume algorithm 60, B-score determined by the B-score algorithm 70, a bone-to-tissue ratio, PPT, and/or PTT, and/or based on the predicted procedure time 2010 or other determinations or outputs 2000 (e.g., assigned staff 2050). The OR layout 2030 may include a room size, a setup, an orientation, starting location, positions and/or a movement or movement path of certain objects or personnel such as robotic device 142, a practitioner, surgeon or other staff member, operating room table, cameras, displays 210, other equipment, sensors, or patient. The procedure time prediction system 10 may determine a series of alerts, warnings, and/or reminders sent to practitioners, hospital staff, and/or patients in preparation for the operation and/or during the operation. The procedure time prediction system 10 may determine or output a new alert to practitioners, hospital staff, and/or patients based on a change in any of the previously determined outputs 2000, which may be based on newly acquired preoperative data 1000 and/or intraoperative data 3000 described later. In some examples, an alert may be a message or indication displayed on a graphical user interface preoperatively or intraoperatively.

For example, the procedure time prediction system 10 may schedule a longer surgery time based on a longer predicted procedure time 2010 (and/or parameters associated with a longer procedure time 2010, such as a narrower joint space width, a larger osteophyte volume, a larger B-score, a deformity, a larger bone-to-tissue ratio, PPT, and/or PTT, etc.), and may determine certain relative positions of staff and/or equipment in the operating room layout 2030 based on determined assigned staff 2050 and/or tools to use as part of the determined procedure plan 2020. The procedure time prediction system 10 may also use surgeon data 1040, planned procedure data 1030, and/or other data (e.g., a hospital's operating room schedule and/or floor plan) to determine an operating room layout 2030 and an operating room schedule 2040. The procedure time prediction system 10 may optimize the OR layout 203 and/or the operating room schedule 2040 to reduce and/or optimize the predicted procedure time 2010. For example, the procedure time prediction system 10 may place certain equipment to clear a movement path for staff and/or for the surgical robot 142 to reduce actual time spent during the procedure. The procedure time prediction system 10 may also determine case management and/or workflow priorities for hospital staff, such as a priority order of case or data processing, based on the other outputs 2000.

The procedure time prediction system 10 may also determine or be used to determine surgeon ergonomics 2070 guidance. For example, the procedure time prediction system 10 may recommend certain postures or positions for assigned staff 2050 based on a longer predicted procedure time 2010 (and/or parameters associated with a longer procedure time 2010, such as a narrower joint space width, a larger osteophyte volume, a larger B-score, a more severe deformity, a larger bone-to-tissue ratio, PPT, and/or PTT, etc.), past experience of the assigned staff 2050, and/or tools to use as part of the determined procedure plan 2020. The procedure time prediction system 10 may optimize surgeon ergonomics 2070 to reduce and/or optimize the predicted procedure time 2010.

The outputs 2000 may be output electronically (e.g., on display 210 and/or a mobile device 220) or printed physically (e.g., on paper, canvas, or film 230 or other materials via a printer). The display 210 may include a plurality of screens and/or graphical user interfaces 250 to output the outputs 2000. For convenience of description, display of the outputs 2000 will be described in connection with an electronic display 210 having a plurality of screens 250.

In some embodiments, the procedure time prediction system 10, or one or more other system, may also determine intra-operative steps and/or workflows. For example, the procedure time prediction system 10 may recommend a particular subset of steps that may optimize the procedure workflow and/or minimize the time necessary to complete the operation. In some embodiments, any of the systems described herein may include a deep learning model (a machine-learning model) with osteophyte data/information to predict intra-operative (intra-op) procedure steps and workflows. A deep learning model may be trained on CT data, including but not limited to CT images or features derived from CT images. The deep learning model may output updates to pre-operative, intra-op, and/or post-operative procedure steps and/or updates to procedure workflows based on real-time data, patient data collected prior to the procedure, and/or prior data from one or more patients with one or more similar conditions to the current patient.

The aforementioned machine-learning model may also incorporate patient information, such as body mass index (BMI), age, gender, or any other type of patient information discussed herein. Such patient information may be encoded in one or more format suitable for processing by the deep learning model. Furthermore, the deep learning model may also incorporate osteophyte data. For example, a deep learning model/machine-learning model may be incorporated into the osteophyte volume algorithm 60, and any of the data discussed herein in relation to the osteophyte volume algorithm 60 may be incorporated into a deep learning model as part of osteophyte volume algorithm 60. For example, as discussed hereinabove, such data may include the location and volume of one or more osteophytes, which may be obtained via CT images or other imaging techniques, as well as additional osteophyte-related parameters. For example, density of osteophytes within a target region may be used by the deep learning model to update a procedure workflow. These osteophyte-related parameters may be determined as described throughout this disclosure, or in some embodiments, by an osteophyte detection module, which may be an integrated component of any of the systems described herein or a separate entity. This osteophyte detection module may use various algorithms or techniques, including but not limited to machine learning, image processing algorithms, or the like, to determine the location and volume of osteophytes from CT images or other types of medical images.

The resulting trained deep learning model, with the integration of CT data, patient information, and osteophyte data, may provide enhanced predictions. These enhanced predictions may include, but are not limited to, the sequence of intra-op steps, the estimated duration of each step, the potential complications that may arise during surgery, or the like. Furthermore, such trained models may also predict the balancing workflow, which may assist in intra-op decision making. The prediction of the balancing workflow may be based on various factors, such as the presence and extent of osteophytes, the patient's BMI, age, gender, or the like.

These factors may be processed by one or more modules or systems described herein, and/or by a balancing workflow prediction module, which may be an integrated component of the system or a separate entity. The balancing workflow prediction module may utilize the output of the trained deep learning model to predict the most appropriate balancing workflow for a given patient, thereby assisting in intra-op decision making. While the above description focuses on deep learning models, other types of machine learning models or statistical models may also be used.

Joint balancing may be performed during a total knee arthroplasty (TKA) procedure, or any other orthopedic procedure, and may be performed prior to resection of the patient (e.g. prior to a tibial cut, etc.), and/or mid-resection or after a first tibial cut or other resection. In some examples, one or more of the systems described herein may determine the presence and/or extend of osteophytes present within a target area, and adjust when joint balancing will occur during a medical procedure. For example, intra-operative updates to a surgical plan may occur mid-resection and may change a surgical plan to include additional joint balancing steps based on intraoperative data, such as intraoperative osteophyte data.

In some examples, ligament integrity may be assessed prior to and/or during a medical procedure. For example, one or more algorithms may use an assessment of ligament integrity, such as how many osteophytes are present on a ligament or the degree of calcification of a ligament, to determine what type of implant to use in a medical procedures. In some examples, one or more algorithms may determine whether to use a posterior stabilizing (PS) implant or a cruciate retaining (CR) implant based on an assessment of ligament integrity or based on any other assessment and/or data discussed herein.

In some examples, as discussed hereinabove, deformity may be determined using CT image data. For example, based on the presence of osteophytes, the amount of coronal deformity correction that can occur due to removal of osteophytes may be predicted by one or more algorithms in one or more systems discussed herein. In some examples, a quantity of osteophyte removal may be determined based on a deformity correction algorithm, which may utilize any of the patient data discussed herein. In some examples, CT image data may indicate one or more flexion contractures, and a surgical plan may be updated to account for the detected flexion contractures. In some examples, a surgical plan may be adjusted to reduce flexion contractures, such as by additional removal of osteophytes, adjustments to resection lengths and angles, and implant selection and size adjustments.

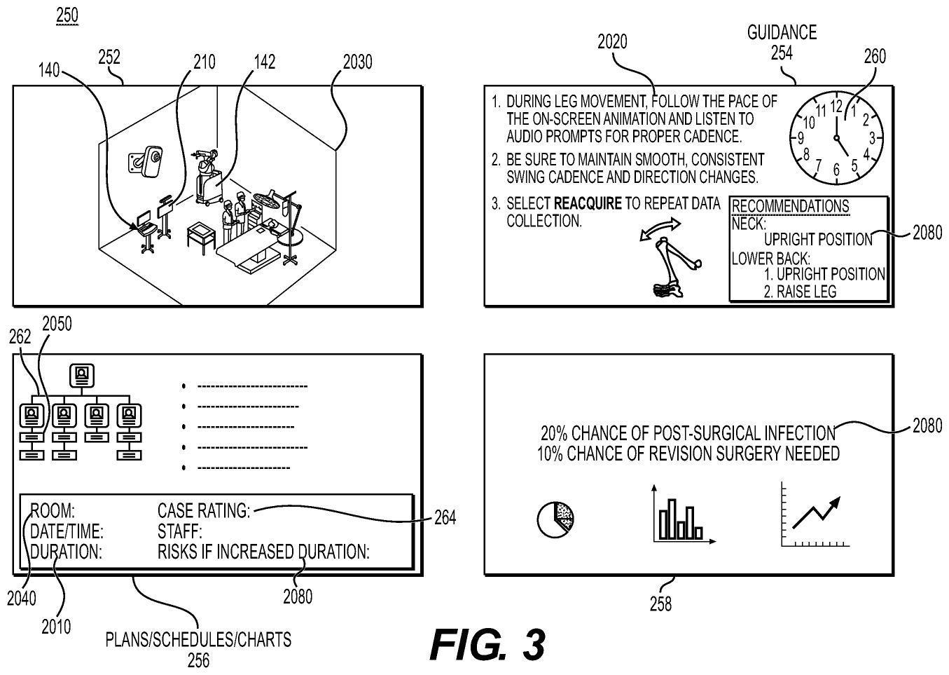

Referring to FIG. 3, a plurality of graphical user interfaces (GUIs) 250 are shown. Each of the graphical user interfaces 250 shown in FIG. 3 may be displayed by itself or at the same time as any one or more other graphical user interfaces 250. The plurality of graphical user interfaces 250 output on the display 210 may include an operating room (OR) layout GUI 252. The OR layout GUI 252 may visually depict a determined OR layout 2030 in a model operating room and/or a simulation of a planned operating room. For example, the OR layout GUI 252 may visually depict relative positions of an operating table and/or bed, a surgical robot 142 (FIG. 2), the display 210 (FIG. 2), staff, tools, lights, sensors, cameras, etc. Alternatively or in addition to a visualization of the OR layout 2030, the OR layout GUI 252 may provide textual instructions and/or descriptions of the OR layout 2030.

The plurality of GUIs 250 may include a guidance GUI 254. The guidance GUI 254 may provide steps or instructions of the procedure plan 2020, instructions for pacing of the procedure plan 2020 in accordance with the predicted procedure time 2010. The guidance GUI 254 may display a clock, stopwatch, and/or timer 260 configured to guide staff through pacing of certain steps in the procedure plan 2020. The guidance GUI 254 may also display animations and/or provide other notifications (e.g., sounds or haptic guidance providing a beat or cadence) to guide staff through pacing. As an example, the guidance GUI 254 may display instructions such as “During leg movement, follow the pace of the on-screen animation and listen to audio prompts for proper cadence.” The procedure time prediction system 10 may determine the pace and/or cadence of prompts for each step of the procedure plan 2020 based on the determined procedure plan 2020, predicted procedure time 2010, and/or other outputs 2000 (e.g., assigned staff 2050 and/or OR schedule 2040). In some examples, the guidance GUI 254 may alert the surgeon and/or staff when a procedure is moving through procedure steps slower than expected and provide an updated total procedure time. The guidance GUI 254 may have other guidance instructions such as “Be sure to maintain smooth, consistent swing cadence and direction changes.”

The guidance GUI 254 may also include recommendations for surgeon ergonomics 2060 along with (or alternatively, on a separate screen as) the steps of the procedure plan 2020. For example, the guidance GUI 254 may display textual recommendations or visual examples of a surgeon's posture, such as “Neck: upright position” and “Lower back: 1. Upright position 2. Raise leg. These recommendations may be determined by the procedure time prediction system 10 to reduce the predicted procedure time 2010. Sequential steps and/or recommendations of the procedure plan 2020 may automatically updated on the guidance GUI 254 and/or may be progressed through a manual input (e.g., by touching a button or the screen of the display 210).

The plurality of GUIs 250 may include an operating schedule GUI 256. The operating schedule GUI 256 may visually depict the assigned staff 2050 in, for example, an organization or staff chart 262, as a list, etc. that identifies or designates individuals to assist in performance of the procedure plan 2020. The operating schedule GUI 256 may also include OR schedule 2040 determinations (e.g., date, time, room number), predicted procedure time 2010, information related to the procedure plan 2020 (e.g., special equipment needed), and a case rating 264 indicating a determined case rating as part of the predicted outcomes 2080. The case rating 264 may indicate how hard or difficult the procedure is determined or predicted to be and/or a level of expertise recommended for the staff for the procedure. The operating schedule screen 258 may also display other predicted outcomes 2080, such as a list of risks (e.g., infection) if the procedure duration exceeds the predicted procedure time 2010.

The plurality of GUIs 250 may also include a predicted outcomes or risks GUI 258 to display predicted outcomes 2080, such as a likelihood of infection after surgery and/or a likelihood of revision surgery. These likelihoods may be correlated to a case rating 264 and/or may be independent from a case rating 264. The likelihoods may be listed as text and/or visually depicted in graphs or charts.

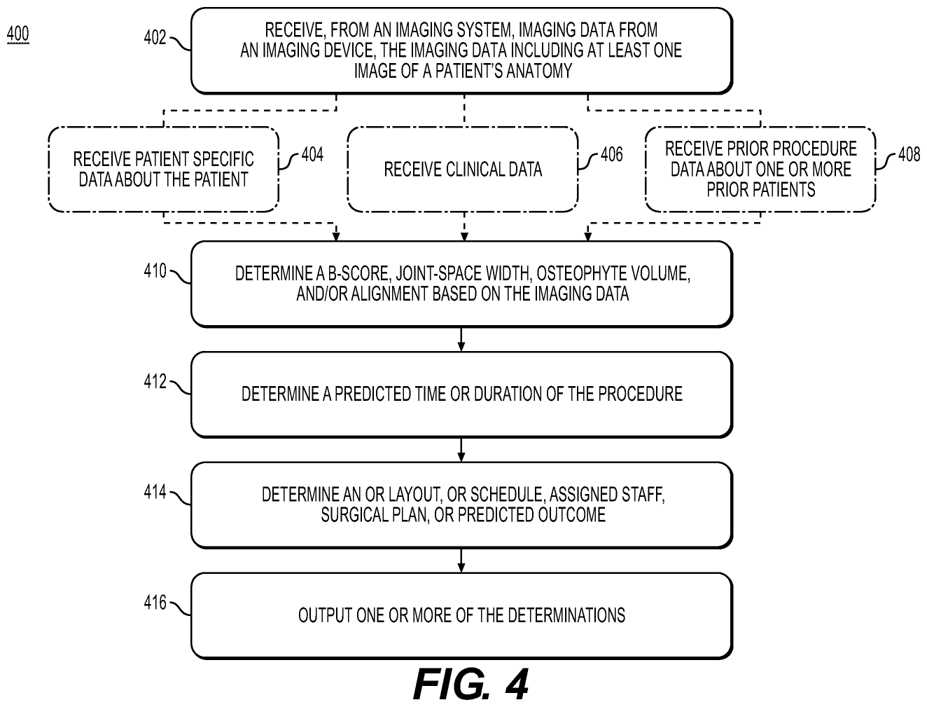

Referring to FIG. 4, an exemplary method 400 according to an embodiment may be used to optimize procedure times and outcomes. The method 400 may include a step 402 of receiving, from an imaging system having an imaging device 110, imaging data 1080. The imaging data 1080 may include at least one image or representation acquired of an instant patient's anatomy (e.g., leg or knee joint). The imaging device 110 may be a CT imaging machine, an MM machine, an x-ray machine, etc. and the image may be a CT scan, an MR scan, an x-ray image, etc. The image may visualize internal structures (e.g., bone and/or tissues) of the instant patient. In step 402, the procedure time prediction system 10 may receive the imaging data 1080 into memory system 20.

The method 400 may also include a step 404 of receiving patient specific data about the instant patient. The patient specific data may include patient data and medical history 1020. For example, the step 404 may include receiving information about patient demographics, biometrics, treatment history, observations, etc. from EMR 120 and/or input (e.g., at an intake appointment) by a practitioner through an interface 130. Step 404 may also include receiving patient information directly from the instant patient using, for example, an application through an interface 130 on a mobile device. In step 404, the procedure time prediction system 10 may store the patient specific data in memory system 20.

The method 400 may also include a step 406 of receiving clinical data, such as information about the planned procedure 1030 and/or surgeon or staff data 1040. The clinical data may be input by a practitioner or other staff into a user interface or application 130 to be received by the procedure time prediction system 10. In step 406, the procedure time prediction system 10 may receive the clinical data into memory system 20.

The method 400 may include a step 408 of receiving prior procedure data 1050 of one or more prior patients. The prior procedure data 1050 may be input by a practitioner and received in memory system 20, or may already be incorporated into the stored data 30 of the memory system 20. The prior patients may share at least one physical characteristic (e.g., demographics, biometrics, disease or disease state, etc.) with the instant patient and may have undergone a similar procedure as the instant patient.

The method 400 may include a step 410 of determining at least one of a B-score, joint-space width, osteophyte volume, and/or alignment or deformity data based on the received imaging data 1080. In step 410, the procedure time prediction system 10 may use one or more algorithms 90 to determine parameters relating to B-score, joint-space width, osteophyte volume, and/or alignment or deformity for at least one bone of interest. For example, the procedure time prediction system 10 may execute a B-score algorithm 70 to determine B-score and related parameters for a femur, a joint-space width algorithm 50 to determine a medial and/or lateral joint-space width between a femur and a tibia, an osteophyte volume algorithm 60 to determine a total osteophyte volume and/or number of osteophytes detected on the femur and tibia, and an alignment/deformity algorithm 80 to determine or detect alignment and/or deformities at the knee joint (e.g., a varus-valgus deformity and/or tilt).

The method 400 may include a step 412 of determining a predicted time or duration 2010 of the procedure to be undergone by the instant patient based on the determined B-score, joint-space width, osteophyte volume, and/or alignment or deformity. In step 412, the procedure time prediction system 412 may determine the procedure time 2010 by executing the one or more algorithms 90 and/or another algorithm based on the outputs by the one or more algorithms 90. The procedure time prediction system 10 may determine a total time of the procedure and also a time, pacing, and/or cadence of one or more steps of the procedure.

The method 400 may include a step 412 of determining, based at least in part on the determined predicted procedure time 2010 and/or the determined B-score, joint-space width, osteophyte volume, and/or alignment or deformity, a procedure plan 2020, an operating room layout 2030, an operating room schedule 2040, and/or predicted outcomes 2080. For example, the procedure time prediction system 10 may determine guidance and/or instructions, such as for keeping a cadence or for performing steps of a procedure, to display on the display 210 as part of determining the procedure plan 2020. The procedure time prediction system 10 may also determine a level of difficulty of a case or the procedure as part of predicted outcomes 2080.

The procedure time prediction system 10 may, based on the determined procedure time 2010 and/or the case difficulty, determine staff members selected from members and data stored in the memory system 20 and/or recommend experience levels or specialties for staff members that perform the procedure. The procedure time prediction system 10 may determine an operating room layout 2030 configured to reduce or optimize the procedure time 2010, such as by configuring a travel path or clearance for staff or a robotic device 142 configured to assist in surgery or other staff and/or determining equipment placement to allow for smooth movement, travel, and/or assistance by the robotic device 142. The procedure time prediction system 10 may also determine risks (e.g., infection risk and/or risks during a procedure, such as complications with blood flow, heart rate, breathing, etc.) related to the procedure based on the determined procedure time 2010 and/or the determined case difficulty.

The method 400 may include in step 416, a step 416 of outputting one or more of the determinations. For example, step 416 may include outputting the predicted procedure time 2010, procedure plan 2020, operating room layout 2030, operating room schedule 2040, assigned staff 2050, surgeon ergonomics 2060, and/or predicted outcomes 2080 on the electronic display 210 using the plurality of screens 250 previously described with reference to FIG. 3. Alternatively or in addition thereto, the determinations may be output audibly via a speaker and/or haptically (e.g., a determined cadence or pacing of a procedure step that is output via vibrations on a robotic tool or device).

Referring to FIG. 5, one or more intraoperative measurement systems 300 may collect (via arrow 302) intraoperative data 3000 during the procedure. During a medical treatment plan or procedure, the procedure time prediction system 10 may collect, receive (e.g., from intraoperative measurement systems 300 via arrow 304), and/or store intraoperative data 3000. The procedure time prediction system 10 may determine intraoperative outputs 4000 and output or send (via arrow 306) the intraoperative outputs 4000 to the output systems 200.

Although the term “intraoperative” is used, the word “operative” should not be interpreted as requiring a surgical operation. Postoperative data may also be collected, received, and/or stored after completion of the medical treatment or medical procedure to become prior procedure data 1050 for a subsequent procedure and/or so that the one or more algorithms 90 may be refined. The intraoperative outputs 4000 may be an updated or refined form of outputs 2000 determined preoperatively (FIG. 2) and/or may be newly generated. The intraoperatively determined outputs 4000 may also be referred to as secondary outputs 4000. Because many of the devices in the one or more intraoperative measurement systems 300 are similar to devices in the one or more preoperative measurement systems 100, many of the types of intraoperative data 3000 are similar to the preoperative data 2000, and many of the processes used and information included in the intraoperative outputs 4000 are similar to those with respect to the preoperatively determined outputs 2000. Any of the preoperative measurement systems 100 and data described herein may also be used and/or collected intraoperatively. Although certain information is described in this specification as being intraoperative data 3000 or intraoperatively determined outputs 4000 and/or postoperative data or postoperatively determined outputs, due to continuous feedback loops of data (which may be anchored by memory system 20), the intraoperative data 3000 described herein may alternatively be determinations or outputs 4000, and the intraoperatively determined outputs 4000 described herein may also be used as inputs into the procedure time prediction system 10. For example, some intraoperative data 3000 may be directly sensed or otherwise received, and other intraoperative data 3000 may be determined, processed, or output based on other intraoperative data 3000, preoperative data 1000, and/or stored data 30.

Like the preoperative measurement systems 100, the intraoperative measurement systems 300 may include electronic medical records and/or user interfaces or applications 340 and imaging devices 350 (e.g., an intraoperative X-ray device or a fluoroscopy device configured for intraoperative use). The intraoperative measurement systems 300 may also include a robot system 310 including a robotic device 142 (e.g., surgical robot), sensors and/or devices 320 to conduct intraoperative tests (e.g., range of motion tests), and sensored implants 330 (e.g., a trial implant). The intraoperatively determined outputs 4000 may include intraoperatively determined (e.g., updated) or secondary procedure time or duration 4010, procedure plan 4020, OR layout 4030, OR schedule 4040, assigned staff 4050, surgeon ergonomics 4070, and/or predicted outcomes 4080.

The user interfaces or applications 340 may be used to input or update procedure information 3030, surgeon data 3040, and staff collected data 3050 (e.g., observations during a procedure and/or other data from sensors that may not have wireless communication modules, such as traditional thermometers). The updated procedure information 3030, surgeon data 3040, and staff collected data 3050 may be updated or refinements to preoperative data 1000 and/or newly generated. The imaging devices 350 may collect imaging data 3080, which may be similar to preoperatively collected imaging data 1080.

The robotic device 142 may be a surgical robot, a robotic tool manipulated or held by the surgeon and/or surgical robot, or other devices configured to facilitate performance of at least a portion of a surgical procedure, such as a joint replacement procedure involving installation of an implant. In some examples, a surgical robot may be configured to automatically perform one or more steps of a procedure. Robotic device refers to surgical robot systems and/or robotic tool systems, and is not limited to a mobile or movable surgical robot. For example, robotic device may refer to a handheld robotic cutting tool, jig, burr, etc.

For convenience of description, the robotic device 142 will be described as a robot configured to move in an operating room and assist staff in performing at least some of the steps of the preoperatively determined procedure plan 2020 and/or a newly generated, refined, or updated procedure plan 4040 (hereinafter referred to as “intraoperatively determined procedure plan 4040”).

The robotic device 142 may include or be configured to hold (e.g., via a robotic arm), move, and/or manipulate surgical tools and/or robotic tools such as cutting devices or blades, jigs, burrs, scalpels, scissors, knives, implants, prosthetics, etc. The robotic device 142 may be configured to move a robotic arm, cut tissue, cut bone, prepare tissue or bone for surgery, and/or be guided by a practitioner via the robotic arm to execute the procedure plan 2020 and/or intraoperatively determined procedure plan 4040. The determined procedure plan 2020 and/or intraoperatively determined procedure plan 4040 may include instructions and/or algorithms for the robotic device 142 to execute.

The robotic device 142 may include and/or use various sensors (pressure sensors, temperature sensors, load sensors, strain gauge sensors, force sensors, weight sensors, current sensors, voltage sensors, position sensors, IMUs, accelerometers, gyroscopes, position sensors, optical sensors, light sensors, ultrasonic sensors, acoustic sensors, infrared or IR sensors, cameras, etc.), sensored tools, cameras, or other sensors (e.g., timer, temperature, etc.) to record and/or collect robot data 3010.

The robot system 310 and/or robotic device 142 may include one or more wheels to move in an operating room, and may include one or more motors configured to spin the wheels and also manipulate surgical limbs (e.g., robotic arm, robotic hand, etc.) to manipulate surgical or robotic tools or sensors. The robotic device 142 may be a Mako SmartRobotics™ surgical robot, a ROBODOC® surgical robot, etc. However, aspects disclosed herein are not limited to mobile robotic devices 142.

The robotic device 142 may be controlled automatically and/or manually (e.g., via a remote control or physical movement of the robotic device 142 or robotic arm by a practitioner). For example, the procedure plan 2020 and/or intraoperatively determined procedure plan 4040 may include instructions that a processor, computer, etc. of the robotic device 142 is configured to execute. The robotic device 142 may use machine vision (MV) technology for process control and/or guidance. The robotic device 142 may have one or more communication modules (WiFi module, BlueTooth module, NFC, etc.) and may receive updates to the procedure plan 2020 and/or intraoperatively determined procedure plan 4040. Alternatively or in addition thereto, the robotic device 142 may be configured to update the procedure plan 2020 and/or generate a new and/or intraoperatively determined procedure plan 4040 for execution.

The robot data 3010 may include data relating to the operating room, movement by staff and/or the robotic device 142, actual time spent on steps of the procedure plan 2020 and/or intraoperatively determined procedure plan 4040, actual total procedure time (e.g., as compared to the determined procedure time 2010). The robotic system 310, via robotic device 142, may also collect or sense information regarding performed procedure steps, such as incision length or depth, bone cut or resection depth, or implant position or alignment. The robotic system 310, via robotic device 142, may also collect or sense information from the patient, such as biometrics pressure, body temperature, heart rate or pulse, blood pressure, breathing information, etc. The robotic system 310 may monitor and/or store information collected using the robotic device 142, and may transmit some of the information after the procedure is finished rather than during the procedure.

The other sensors and/or devices 320 may include one or more sensored surgical tools (e.g., a sensored marker), wearable tools, sensors, or pads, etc. The sensors and/or devices 320 may be applied to or be worn by the patient during the execution of procedure plan 2020 and/or intraoperatively determined procedure plan 4040, such as a wearable sensor, a surgical marker, a temporary surgical implant, etc. Although some sensors and/or devices 320 may also be sensored implants 330 or robotic devices 142 (e.g., robotic surgical tools configured to execute instructions and/or use feedback from sensors using motorized tool heads), other sensors and/or devices 320 may not strictly be considered an implant or a robotic device. For example, the sensors and/or devices 320 may be or include a tool (e.g., probe, knife, burr, etc.) used by medical personnel and including one or more optical sensors, load sensors, load cells, strain gauge sensors, weight sensors, force sensors, temperature sensors, pressure sensors, etc.

The procedure time prediction system 10 may use the sensors and/or devices 320 to collect sensored data 3100, which may include pressure, incision length and/or position, soft tissue integrity, biometrics, etc. In addition, the sensored data 3100 may include alignment data 3020, range of motion data (e.g., collected during intraoperative range of motion tests by a practitioner manipulating movement at or about the joints) and/or kinematics data.

The one or more sensored implants 320 may include temporary or trial implants applied during the procedure and removed from the patient later during the procedure and/or permanent implants configured to remain for postoperative use. The one or more sensored implants 320 may include implant systems for a knee (e.g., femoral and tibial implant having a tibial stem, sensors configured to be embedded in a tibia and/or femur), hip (e.g., femoral implant having a femoral head having an acetabular component and/or stem), shoulder (e.g., humeral or humerus implant), spine (e.g., spinal rod or spinal screws), or other joint or extremities implants, replacements, prosthetics (e.g., fingers, forearms, etc.). The sensored implants 320 may include one or more load sensors, load cells, force sensors, weight sensors, current sensors, voltage sensors, position sensors, IMUs, accelerometers, gyroscopes, optical sensors, light sensors, ultrasonic sensors, acoustic sensors, infrared or IR sensors, cameras, pressure sensors, temperature sensors, etc.

The sensored implants 320 may collect sensored data 3100 and/or alignment data 3080, such as range of motion, pressure, biometrics, implant position or alignment, implant type, design, or material, etc. The sensored implants 320 may also be configured to sense and/or monitor infection information (e.g., by sensing synovial fluid color or temperature).

The intraoperative measurement systems 300 is not limited to the sensors discussed herein. For example, intraoperative data 3000 may also be collected using cameras or motion sensors installed in an operating room (e.g., camera above an operating table, high up on a wall, or on a ceiling) or a sensored patient bed or operating table (e.g., having temperature sensors, load cells, pressure sensors, position sensors, accelerometers, IMUs, timers, clocks, etc. to collect information on an orientation or position of the patient and biometrics, heart rate, breathing rate, skin temperature, skin moisture, pressure exerted on the patient's skin, patient movement/activity, etc., movement or position of the bed or table via wheel sensors, and/or a duration of the procedure). In addition, the intraoperative data 3000 may include prior procedure data 3090 from prior procedures with similar patients and/or similar intraoperative data 3000. The intraoperative data 3000 may include the same types of data in preoperative data 1000 and/or data such as operating room efficiency and/or performance, tourniquet time, blood loss, biometrics, incision length, resection depth, soft tissue integrity, pressure, range of motion or other kinematics, implant position or alignment, and implant type or design, though this list is not exhaustive.

As another example, cameras and/or a navigational system may be used to track operating room efficiency, pacing, layout information, information on staff and/or surgeon's performing the procedure plan 2020 and/or intraoperatively determined procedure plan 4020, and/or movement and posture patterns (measured by, for example, wearable sensors, external sensors, cameras and/or navigational systems, surgical robot 142, etc.) Based on intraoperatively collected data 3000, the procedure time prediction system 10 may determine, in determining surgeon ergonomics 4070, that a table is too high for a surgeon and determine a lower height for the table in an updated operating room layout 4030, which may increase operating room efficiency and thus decrease a determined procedure duration 4010 and may reduce fatigue for a surgeon working over the operating table.

The procedure time prediction system 10 may execute the one or more algorithms 90 to determine intraoperative outputs 4000 based on the intraoperative data 3000 similarly to how the one or more algorithms determined outputs 2000 based on the preoperative data 1000. The one or more algorithms 90 may also determine the intraoperative outputs 4000 based on the previously collected and/or stored intraoperative data 1000 and any other stored data 30, such as prior procedure data 3090. For example, the joint-space width algorithm 50 may use intraoperative data 3000 to determine, intraoperatively, joint space width dimensions, such as an updated joint space width between two bones based on intraoperative data 3000 and/or a new joint space width when an implant (e.g., trail implant 330 and/or permanent implant 330) is applied or other corrective steps in the procedure are performed. The osteophyte volume algorithm 60 may determine osteophyte position and volume, such as an updated position and volume based on intraoperative data 3000 and/or a new position and volume after certain steps in the procedure are performed, such as when bone cuts are made. The B-score algorithm 70 may determine an updated B-score based on intraoperative data 3000 and/or a new B-score based on when an implant is applied or when other corrective steps in the procedure are performed. The alignment/deformity algorithm 80 may determine updated alignment and deformity information of the patient's bones based on intraoperative data 3000 and/or new alignment and deformity information after an implant is applied or certain steps of the procedure are performed.

Like outputs 2000 determined preoperatively, the intraoperative outputs 4000 may include surgical time 4010, procedure plan 4020, operating room layout 4030, operating room schedule 4040, assigned staff 4050, surgeon ergonomics 4070, and predicted outcomes 4080. As an example, based on complications during the procedure or due to certain information (e.g., alignment, deformity, or infection) that is more readily apparent intraoperatively once a tissue cut has been made, the procedure time prediction system 10 may determine, intraoperatively, an increase in procedure time 4010, an increase in an amount of time left in procedure time 4010, and/or a new surgical time 4010 longer than preoperatively determined procedure time 2010. These intraoperative outputs 4000 may be output on the previously described output systems 200.

The longer procedure time 4010 may affect the other intraoperative outputs 4000. For example, the procedure time prediction system 10 may determine that the procedure plan 4020 should include adjusted or extra steps, that an operating room layout 4030 should be adjusted, that the operating room schedule 4040 should be adjusted (and/or that other bookings using some same staff members or a same room should be adjusted) that the assigned staff 4050 should include more or less staff members, that surgeon ergonomics 4070 should include positions suited to the longer duration, and that the predicted outcomes 4080 may include higher risks for postoperative infection, higher perceived pain, higher stress level, higher anxiety level, lower mental health status higher cartilage loss, and/or increase the case difficulty.

Similarly, based on a pacing of the procedure by the assigned staff, the procedure time prediction system 10 may predict an increase or decrease in procedure time 4010. In the case where the procedure time prediction system 10 predicts an increase in procedure time 4010 due to pacing rather than complications (e.g., infections), the procedure time prediction system 10 may determine new pacing of steps in the procedure plan 4020 and/or new guidances to output on display 210 to catch the surgeon up and possibly get the timing back on track. In the case where the procedure time prediction system 10 determines a shorter procedure time 4010 due to pacing, the procedure time prediction system 10 may determine new pacing of steps in the procedure plan 4020 and/or new guidances to output on display 210 to slow the surgeon down and possibly get the timing back on track. Alternatively or in addition thereto, the procedure time prediction system 10 may determine that the procedure plan 4020 should include adjusted or extra steps, that an operating room layout 4030 should be adjusted, that the operating room schedule 4040 and/or a cleaning time should be adjusted, that the assigned staff 4050 should include more or less staff members, that surgeon ergonomics 4070 should include positions suited to the shorter duration, and that the predicted outcomes 4080 may include lower risks for postoperative infection, lower perceived pain, lower stress level, lower anxiety level, and/or higher mental health status, lower cartilage loss, and/or decrease the case difficulty.

In some cases, the procedure time prediction system 10 may determine that the procedure should be stopped and/or postponed for a later date based on extreme complications of a patient's alignment and/or infection status and/or external factors (e.g., other emergencies at an institution, weather emergencies, etc.), in which case, the procedure time prediction system 10 may predict a much shorter procedure time 4010 based on a recommendation to stop and/or postpone the procedure.

The intraoperative measurement systems 300 may periodically and/or continuously sense or collect intraoperative data 3000 (arrow 302), some or all of which may be periodically and/or continuously sent to the procedure time prediction system (arrow 304). The procedure time prediction system 10 may periodically or continuously determine the intraoperatively determined outputs 4000 to update information and may periodically and/or continuously send the intraoperatively determined outputs 4000 to the output systems (arrow 306).

The procedure time prediction system 4000 may periodically and/or continuously compare the predicted outcome data 4080 with target or desired outcomes, and further determine, update, or refine the procedure duration 4010, the procedure plan 4020, and/or other outputs 4000 (e.g., OR layout 4030, OR schedule 4040, assigned staff 4050, and surgeon ergonomics 4070) based on the comparison. The procedure time prediction system 4000 may be configured to output this comparison (e.g., via information and/or visually) to the output system 200, such as the one or more GUIs 250 of the displays 210.

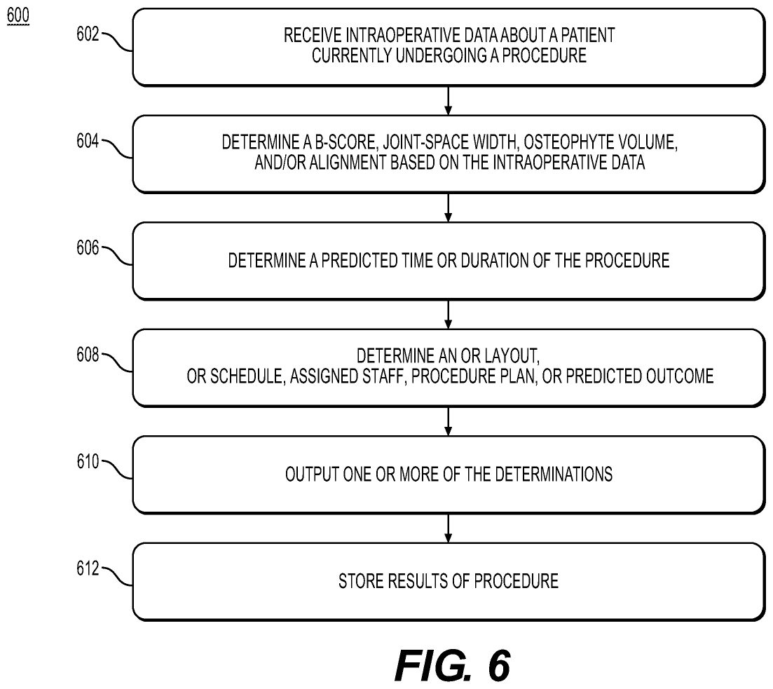

Referring to FIG. 6, an exemplary method 600 according to an embodiment may be used to optimize procedure times and outcomes. The method 600 may be performed in combination with (e.g., after) method 400 and/or in place of method 400. The method 600 may include a step 602 of receiving, from an intraoperative measurement systems 300, intraoperative data 3000. In step 602, the procedure time prediction system 10 may receive the intraoperative data 3000 into memory system 20. In step 602, the procedure time prediction system 10 may also receive preoperative data 1000, prior procedure data, etc.

The method 600 may include a step 604 of determining at least one of a B-score, joint-space width, osteophyte volume, and/or alignment or deformity data based on the received intraoperative data 1000. In step 604, the procedure time prediction system 10 may use one or more algorithms 90 to determine parameters relating to B-score, joint-space width, osteophyte volume, and/or alignment or deformity for at least one bone of interest. For example, the procedure time prediction system 10 may execute a B-score algorithm 70 to determine B-score and related parameters for a femur, a joint-space width algorithm 50 to determine a medial and/or lateral joint-space width between a femur and a tibia, an osteophyte volume algorithm 60 to determine a total osteophyte volume and/or number of osteophytes detected on the femur and tibia, and an alignment/deformity algorithm 80 to determine or detect alignment and/or deformities at the knee joint (e.g., a varus-valgus deformity and/or tilt). Here, the parameters relating to B-score, joint-space width, osteophyte volume, and/or alignment or deformity may be different from parameters determined or stored preoperatively.

The method 600 may include a step 606 of determining a predicted time or duration 4010 of the procedure to be undergone by the instant patient based on the determined B-score, joint-space width, osteophyte volume, and/or alignment or deformity. In step 606, the procedure time prediction system 10 may determine the procedure time 4010 by executing the one or more algorithms 90 and/or another algorithm based on the outputs by the one or more algorithms 90. The procedure time prediction system 10 may determine a total time of the procedure, a time left of the procedure, a change in time of the procedure, and/or a time, pacing, and/or cadence of each individual step (e.g., each step remaining) of the procedure.

The method 600 may include a step 608 of determining, based at least in part on the determined predicted procedure time 4010 and/or the determined B-score, joint-space width, osteophyte volume, and/or alignment or deformity, a procedure plan 4020, an operating room layout 4030, an operating room schedule 4040, and/or predicted outcomes 4080. For example, the procedure time prediction system 10 may determine guidance and/or instructions, such as for keeping a cadence or for performing steps of a procedure, to display on the display 210 as part of determining the procedure plan 4020. The procedure time prediction system 10 may also determine a level of difficulty of a case or the procedure as part of predicted outcomes 4080.

The procedure time prediction system 10 may, based on the determined procedure time 4010 and/or the case difficulty, determine staff members to call into the operating room selected from members and data stored in the memory system 20 and/or recommend experience levels or specialties for staff members that perform and/or assist with the procedure. The procedure time prediction system 10 may determine an operating room layout 4030 configured to reduce or optimize the procedure time 4010, such as by configuring a travel path or clearance for staff or a robotic device 142 configured to assist in surgery or other staff, and/or determining equipment placement to allow for smooth movement, travel or assistance by the robotic device 142. The procedure time prediction system 10 may also determine risks (e.g., infection risk and/or risks during a procedure, such as complications with blood flow, heart rate, breathing, etc.) related to the procedure based on the determined procedure time 4010 and/or the determined case difficulty.

The method 600 may include in step 610, a step 610 of outputting one or more of the determinations. For example, step 610 may include outputting the predicted procedure time 4010, procedure plan 4020, operating room layout 4030, operating room schedule 4040, assigned staff 4050, surgeon ergonomics 4060, and/or predicted outcomes 4080 to the electronic display 210 using the plurality of screens 250 previously described with reference to FIG. 3. Alternatively or in addition thereto, the determinations may be output audibly via a speaker and/or haptically (e.g., a determined cadence or pacing of a procedure step that is output via vibrations on a robotic tool or device). Step 610 of outputting the one or more determinations may also include storing the one or more determinations (e.g., in memory system 20).

The method 600 may include repeating steps 604, 606, 608, and/or 610 throughout a duration of the procedure. The method 600 may include, in step 612, storing results of the procedure, which may become prior procedure data 1050 and/or 3090 in a future procedure.

Although not shown, postoperative data, including actual results 12 (FIG. 1), may be collected by postoperative measurement systems (e.g., user interfaces and/or questionnaires, practitioner-input assessments, wearable sensors, mobile devices, sensored implants, etc.), which may be stored in the memory system 20 as prior procedure data 1040 and/or 3090 and/or be used to determine a procedure time for a future procedure (e.g., a revision procedure). Postoperative data may include information on actual patient outcomes 12 and/or success of surgery, a patient's postoperative lifestyle, patient satisfaction, postoperative clinical data, rehabilitation and/or physical therapy data, planned procedures (e.g., revisions), psychosocial data, postoperative bone imaging, bone density, biometrics, and kinematics including range of motion and/or alignment, postoperative medical history, and recovery. Patient outcomes may include both immediate and long term results and/or metrics from the medical procedure (e.g., surgery). As an example, the one or more algorithms 90 may be configured to analyze patient outcomes and/or actual outcomes 12 to make determinations, such as a success metric or an indication of whether the procedure was successful, changes in joint-space width, osteophyte volume, B-score, alignment/deformity, range of motion, stability, fall risk, fracture risk, joint stiffness or flexibility, or other changes between preoperative data 1000, intraoperative data 3000, and/or postoperative data etc. Patient satisfaction may be a patient-reported (or, alternatively or in addition thereto, a practitioner-reported) satisfaction with the procedure, both immediate and long-term. Medical history information may be updated and may include both immediate and long term information such as new utilization of orthotics, care information in a supervised environment such as a skilled nursing facility or SNF, infection information, etc. Information on recovery may also be included and may include information on adherence to a postoperative or rehabilitative plan such as actual exercises performed, medicine dosage and/or type actually taken, fitness information, planned physical therapy (PT), adherence to PT, etc. Discharge and/or length of stay information may also be collected. This list, however, is not exhaustive and postoperative data may include other patient specific information and/or other inputs manually input by a practitioner. Some of the postoperative data may be directly sensed, and other postoperative data may be determined based on directly sensed or input information. The postoperative data may be stored in the memory system 20 and become prior procedure data 1050 in a future procedure and be used to refine the one or more algorithms 90.

Aspects disclosed herein may use one or more algorithms 90 to analyze one or more CT scans to identify bones (e.g., based on bone landmarks), detect osteophytes, determine an osteophyte volume or related parameters (e.g., positions, a total osteophyte volume, individual osteophyte volume, etc.), and predict a procedure duration based on the determined osteophyte volume or related parameters.

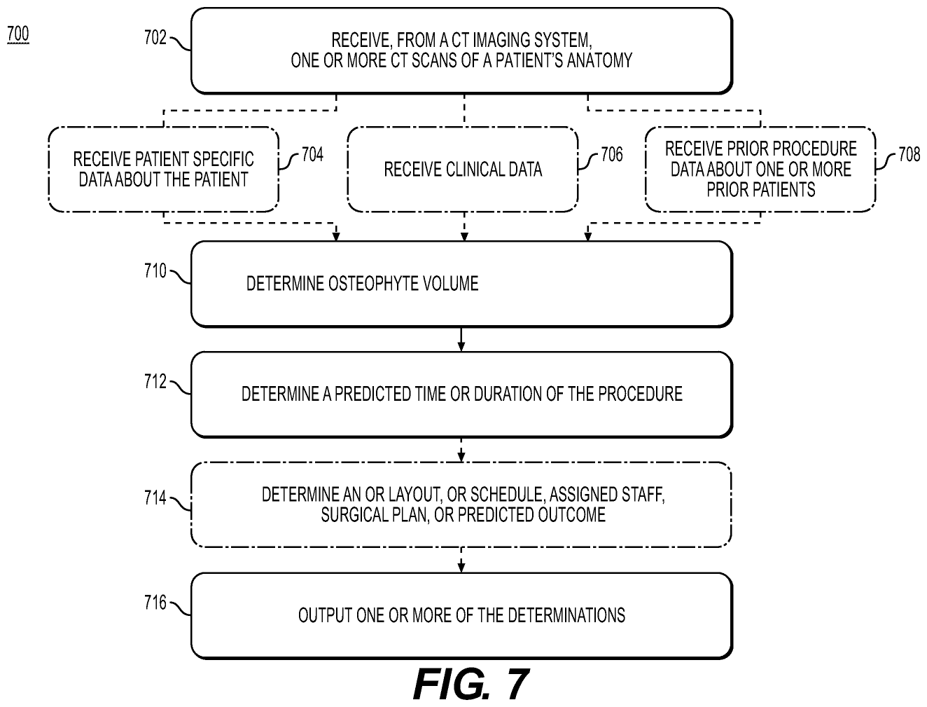

Referring to FIG. 7, an exemplary method 700 according to an embodiment may be used to optimize procedure times and outcomes based on osteophyte volume determined from CT scans. The method 700 may include a step 702 of receiving, from a CT imaging device or imaging system, one or more CT scans, which may be a kind of imaging data 1080. The one or more CT scans may include at least one image or representation acquired of an instant patient's anatomy (e.g., leg or knee joint). The image may visualize internal structures (e.g., bone and/or tissues) of the instant patient. In step 702, the procedure time prediction system 10 may receive raw CT scans into the memory system 20. As an example, the method 700 may include a step 703 of receiving a plurality of CT scans of various viewpoints of a same joint (e.g., anterior, posterior, and side views around a knee joint).

The method 700 may also include a step 704 of receiving patient specific data about the instant patient. The patient specific data may include patient data and medical history 1020. For example, the step 704 may include receiving information about patient demographics, biometrics, treatment history, observations, etc. from EMR 120 and/or input (e.g., at an intake appointment) by a practitioner through an interface 130. Step 704 may also include receiving patient information directly from the instant patient using, for example, an application through an interface 130 on a mobile device. In step 704, the procedure time prediction system 10 may store the patient specific data in memory system 20.

The method 700 may also include a step 706 of receiving clinical data, such as information about the planned procedure 1030 and/or surgeon or staff data 1040. The clinical data may be input by a practitioner or other staff into a user interface or application 130 to be received by the procedure time prediction system 10. In step 706, the procedure time prediction system 10 may receive the clinical data into memory system 20.

The method 700 may include a step 708 of receiving prior procedure data 1050 of one or more prior patients. The prior procedure data 1050 may be input by a practitioner and received in memory system 20, or may already be incorporated into the stored data 30 of the memory system 20. The prior patients may share at least one physical characteristic (e.g., demographics, biometrics, disease or disease state, etc.) with the instant patient and may have undergone a similar procedure as the instant patient.

The method 700 may include a step 710 of determining osteophyte volume based on the one or more received CT scans. In step 710, the procedure time prediction system 10 may use one or more algorithms 90, such as the osteophyte volume algorithm 60, to identify, detect, and/or recognize one or more bones, and to identify, detect, and/or recognize osteophytes on the identified bones. The procedure time prediction system 10 may determine a location and/or position of the detected osteophytes, a total number of osteophytes, and also determine a size and/or volume of the detected osteophytes. The procedure time prediction system 10 may determine an individual volume for each detected osteophyte and/or a total volume of all detected osteophytes. The procedure time prediction system 10 may determine anatomical compartments of the detected osteophytes and determine a total number of osteophytes and/or a total volume of osteophytes in each anatomical compartment. The procedure time prediction system 10 other parameters relating to osteophyte volume and position. As an example, intercondylar notch osteophytes may be indicative of posterior cruciate ligament (PCL) insufficiency, and a surgical plan may be updated to require a posterior stabilizing implant instead of a cruciate retaining implant, which may then adjust the predicted surgical time. In some examples, posterior femoral osteophytes may be correlated to the flexion-extension corrections required during surgery, which may adjust the predicted surgical time. Medial and lateral femoral osteophytes may be correlated to coronal deformity and the ability to correct the deformity in the knee, which may adjust the predicted surgical time based on the volume of medial and lateral femoral osteophytes.

The method 700 may include a step 712 of determining a predicted time or duration 2010 of the procedure to be undergone by the instant patient based on the determined osteophyte volume. In step 712, the procedure time prediction system 712 may determine the procedure time 2010 by executing the one or more algorithms 90 (e.g. osteophyte volume algorithm 60), and/or another algorithm based on the outputs by the one or more algorithms 90. The procedure time prediction system 10 may determine a total time of the procedure and also a time, pacing, and/or cadence of one or more steps of the procedure.

The method 700 may include a step 712 of determining, based at least in part on the determined predicted procedure time 2010 and/or the determined osteophyte volume, a procedure plan 2020, an operating room layout 2030, an operating room schedule 2040 (e.g., staff assignments), and/or predicted outcomes 2080. For example, the procedure time prediction system 10 may determine guidance and/or instructions, such as for keeping a cadence or for performing steps of a procedure, to display on the display 210 as part of determining the procedure plan 2020. The procedure time prediction system 10 may also determine a level of difficulty of a case or the procedure as part of predicted outcomes 2080.

The procedure time prediction system 10 may, based on the determined procedure time 2010 and/or the case difficulty, determine staff members selected from members and data stored in the memory system 20 and/or recommend experience levels or specialties for staff members that perform the procedure. The procedure time prediction system 10 may determine an operating room layout 2030 configured to reduce or optimize the procedure time 2010, such as by configuring a travel path or clearance for staff or a robotic device 142 configured to assist in surgery or other staff and/or determining equipment placement to allow for smooth movement, travel, and/or assistance by the robotic device 142. The procedure time prediction system 10 may also determine risks (e.g., infection risk and/or risks during a procedure, such as complications with blood flow, heart rate, breathing, etc.) related to the procedure based on the determined procedure time 2010 and/or the determined case difficulty.

The method 700 may include a step 716 of outputting one or more of the determinations. For example, step 716 may include outputting the determined osteophyte volume, predicted procedure time 2010, procedure plan 2020, operating room layout 2030, operating room schedule 2040, assigned staff 2050, surgeon ergonomics 2060, and/or predicted outcomes 2080 on the electronic display 210 using the plurality of screens 250 previously described with reference to FIG. 3. Alternatively or in addition thereto, the determinations may be output audibly via a speaker and/or haptically (e.g., a determined cadence or pacing of a procedure step that is output via vibrations on a robotic tool or device).

Aspects disclosed herein may be used to sense or collect preoperative, intraoperative, and/or postoperative information about a patient and/or a procedure.

Aspects disclosed herein contemplate implants or prosthetics, and are not limited to the contexts described. For example, implants disclosed herein may be implemented as another implant system for another joint or other part of a musculoskeletal system (e.g., hip, knee, spine, bone, ankle, wrist, fingers, hand, toes, or elbow) and/or as sensors configured to be implanted directly into a patient's tissue, bone, muscle, ligaments, etc. Each of the implants or implant systems may include sensors such as inertial measurement units, strain gauges, accelerometers, ultrasonic or acoustic sensors, etc. configured to measure position, speed, acceleration, orientation, range of motion, etc. In addition, each of the implants or implant systems may include sensors that detect changes (e.g., color change, pH change, etc.) in synovial fluid, blood glucose, temperature, or other biometrics and/or may include electrodes that detect current information, ultrasonic or infrared sensors that detect other nearby structures, etc. to detect an infection, invasion, nearby tumor, etc. In some examples, each of the implants and/or implant systems may include a transmissive region, such as a transparent window on the exterior surface of the prosthetic system, configured to allow radiofrequency energy to pass through the transmissive region. The IMU may include three gyroscopes and three accelerometers. The IMU may include a micro-electro mechanical (MEMs) integrated circuit. Implants and/or implant systems disclosed herein may also be implemented as implantable navigation systems. For example, the implants may have primarily a sensing function rather than a joint replacement function. The implants may, for example, be a sensor or other measurement device configured to be drilled into a bone, another implant, or otherwise implanted in the patient's body.

The implants, implant systems, and/or measurement systems disclosed herein may include strain gauge sensors, optical sensors, pressure sensors, load cells/sensors, ultrasonic sensors, acoustic sensors, resistive sensors including an electrical transducer to convert a mechanical measurement or response (e.g., displacement) to an electrical signal, and/or sensors configured to sense synovial fluid, blood glucose, heart rate variability, sleep disturbances, and/or to detect an infection. Measurement data from an IMU and/or other sensors may be transmitted to a computer or other device of the system to process and/or display alignment, range of motion, and/or other information from the IMU. For example, measurement data from the IMU and/or other sensors may be transmitted wirelessly to a computer or other electronic device outside the body of the patient to be processed (e.g. via one or more algorithms) and displayed on an electronic display.

Aspects and systems disclosed herein may make determinations based on images or imaging data (e.g., from CT scans). Images disclosed herein may display or represent bones, tissues, or other anatomy, and systems and aspects disclosed herein may recognize, identify, classify, and/or determine portions of anatomy such as bones, cartilage, tissue, and bone landmarks, such as each specific vertebra in a spine. Aspects and systems disclosed herein may determine relative positions, orientations, and/or angles between recognize bones, such as a Cobb angle, an angle between a tibia and a femur, and/or other alignment data.

Aspects and systems disclosed herein provide displays having graphical user interfaces configured to graphically display data, determinations, and/or steps, targets, instructions, or other parameters of a procedure, including preoperatively, intraoperatively, and/or postoperatively. Figures, illustrations, animations, and/or videos displayed via user interfaces may be recorded and stored on the memory system.

Aspects and systems disclosed herein may be implemented using machine learning technology. One or more algorithm may be configured to learn or be trained on patterns and/or other relationships across a plurality of patients in combination with preoperative information and outputs, intraoperative information and outputs, and postoperative information and outputs. The learned patterns and/or relationships may refine determinations made by one or more algorithms and/or also refine how the one or more algorithms are executed, configured, designed, or compiled. The refinement and/or updating of the one or more algorithms may further refine displays and/or graphical user interfaces (e.g., bone recognition and/or determinations, targets, recognition and/or display of other conditions and/or bone offsets, etc.).

Aspects disclosed herein may be configured to optimize a “fit” or “tightness” of an implant provided to a patient during a medical procedure based on detections by the one or more algorithms. A fit of the implant may be made tighter by aligning the implant with a shallower bone slope and/or determining a shallower resulting or desired bone slope, by increasing a thickness or other dimensions of the implant, by determining certain types of materials or a type of implants or prosthesis (e.g., a stabilizing implant, a VVC implant, an ADM implant, or an MDM implant). A thickness of the implant may be achieved by increasing (or decrease) a size or shape of the implant. Tightness may be impacted by gaps and/or joint space width, which may be regulated by an insert which may vary depending on a type of implant or due to a motion. Gaps may be impacted by femoral and tibial cuts. Tightness may further be impacted by slope. A range of slope may be based on implant choice as well as surgical approach and patient anatomy. A thickness of the implant may also be achieved by adding or removing an augment or shim. For example, augments or shims may be stackable and removable, and a thickness may be increased by adding one or more augments or shims or adding an augment or shim having a predetermined (e.g., above a certain threshold) thickness. Fit or tightness may also be achieved with certain types of bone cuts, bone preparations, or tissue cuts that reduce a number of cuts made and/or an invasiveness during surgery.

Aspects disclosed herein may be implemented during a robotic medical procedure using a robotic device. Aspects disclosed herein are not limited to specific scores, thresholds, etc. that are described. For example, outputs and/or scores disclosed herein may include other types of scores such as HOOS, KOOS, SF-12, SF-36, Harris Hip Score, etc.

Aspects disclosed herein are not limited to specific types of surgeries and may be applied in the context of osteotomy procedures, computer navigated surgery, neurological surgery, spine surgery, otolaryngology surgery, orthopedic surgery, general surgery, urologic surgery, ophthalmologic surgery, obstetric and gynecologic surgery, plastic surgery, valve replacement surgery, endoscopic surgery, and/or laparoscopic surgery.

Aspects disclosed herein may improve or optimize surgery durations and outcomes. Aspects disclosed herein may augment the continuum of care to optimize post-operative outcomes for a patient. Aspects disclosed herein may recognize or determine previously unknown relationships, to help optimize care, procedure or surgical time, and/or design of a prosthetic.

What is claimed is:

1. A method for determining a duration of a medical procedure, comprising:
receiving imaging data including at least one image acquired of a patient's anatomy;
determining at least one parameter of the patient's anatomy based on the imaging data, the at least one parameter including at least one of a B-score, a joint-space width, an osteophyte position or volume, an alignment, or a deformity based on the imaging data;
predicting a duration for the medical procedure based on the determined at least one parameter; and
outputting the predicted duration on an electronic display.

2. The method of claim 1, further comprising:
identifying at least one femur in the at least one image, wherein the parameter includes a B-score of the identified femur;
determining that the B-score is greater than a predetermined B-score, and
determining that the predicted duration is longer or shorter than a predetermined duration.

3. The method of claim 1, further comprising:
identifying at least two bones at a joint in the at least one image, wherein the parameter includes a joint-space width between the at least two bones; and
determining whether the joint-space width is within a predetermined joint-space width range.

4. The method of claim 3, further comprising:
determining that the joint-space width is outside the predetermined joint-space width range, and
determining that the predicted duration is longer than a predetermined duration.

5. The method of claim 1, further comprising:
identifying at least one bone in the at least one image; and
detecting at least one osteophyte on the identified at least one bone.

6. The method of claim 5, further comprising:
determining a volume of the detected at least one osteophyte, and
determining that the predicted duration is longer or shorter than a predetermined duration based on the determined volume.

7. The method of claim 5, wherein detecting at least one osteophyte on the identified at least one bone includes determining a position of the at least one osteophyte in relation to a predetermined area or compartment on the identified bone.

8. The method of claim 1, further comprising:
identifying at least one bone in the at least one image;
determining an alignment parameter of the at least one bone; and
determining whether the alignment parameter is within a predetermined alignment range.

9. The method of claim 8, further comprising:
determining that the alignment parameter is outside the predetermined alignment range, and
determining that the predicted duration is longer than a predetermined duration.

10. The method of claim 1, further comprising:
receiving prior procedure data, the prior procedure data including data from a plurality of prior patients sharing at least one characteristic with the patient, wherein determining the predicted duration for the medical procedure is based on the received prior procedure data.

11. The method of claim 1, further comprising:
receiving at least one of (i) patient specific data regarding the patient, (ii) clinical data relating to the patient, and (iii) surgeon specific data relating to one or more surgeons, wherein determining the predicted duration for the medical procedure is based on the received patient specific data, clinical data, and/or surgeon specific data.

12. The method of claim 1, further comprising determining, based on the determined predicted duration for the procedure and/or the at least one parameter of the patient's anatomy, an output, the output including at least one of:
an operating room layout,
an operating room schedule,
at least one staff member to assist in performance of the medical procedure,
a procedure plan,
a case difficulty,
a risk of infection,
a loss of cartilage,
a predicted pain perceived by the patient after the procedure,
a predicted stress level perceived by the patient after the procedure,
a predicted anxiety level perceived by the patient after the procedure, or
a predicted mental health status of the patient after the procedure.

13. The method of claim 12, wherein determining the output includes determining the operating room layout, the operating room schedule, and the at least one staff member, and the determined output is configured to reduce the duration for the procedure.

14. The method of claim 1, further comprising determining, based on the predicted procedure duration, at least one of a case difficulty, a risk of infection, a loss of cartilage, or a predicted pain, stress level, anxiety level, or mental health status of the patient.

15. The method of claim 1, further comprising determining, based on the imaging data, at least one of a bone-to-skin ratio and a bone-to-tissue ratio, wherein predicting the duration for the medical procedure is based on the determined bone-to-skin ratio and/or bone-to-tissue ratio.

16. The method of claim 1, further comprising:
receiving procedure information collected during the medical procedure; and
determining a secondary duration for the medical procedure based on the received procedure information.

17. A method for determining a duration for a medical procedure, comprising:
receiving at least one image acquired of a patient's anatomy;
determining, based on the at least one image, a plurality of parameters, the plurality of parameters including: (i) a B-score, (ii) a joint-space width, (iii) an osteophyte position or volume, and (iv) an alignment or a deformity relating to the patient's anatomy;
predicting a duration for the medical procedure based on the determined plurality of parameters, and
outputting the predicted duration on an electronic display.

18. The method of claim 17, wherein predicting the duration includes determining a longer duration of the medical procedure based on:
a determined B-score that is outside a predetermined B-score range,
a determined joint-space width that is outside a predetermined joint-space width range,
a determined osteophyte volume that is outside a predetermined osteophyte volume range, and/or
a determined misalignment or severity of the deformity that is outside of a predetermined alignment range.

19. A system configured to predict a duration for a medical procedure, comprising:
an imaging device configured to acquire at least one image of a patient's anatomy;
a memory configured to store information, the information including patient specific information, clinical data, practitioner specific information, preoperative data received from one or more preoperative measurement systems, and prior procedure data related to prior patients that underwent prior procedures;
a controller configured to:
execute one or more algorithms to determine, based on the at least one image, at least one parameter of the patient's anatomy, the parameter including at least one of a B-score, a joint-space width, an osteophyte position or volume, an alignment, and a deformity,
determine, based on the determined at least one parameter and the stored information in the memory, a duration of the medical procedure to be undergone by a patient, and
determine, based on the predicted duration, an output including at least one of an operating room layout, an operating room schedule, at least one staff member to assist in performance of the procedure, a procedure plan, a case difficulty, a risk of infection, a loss of cartilage, or a predicted pain, stress level, anxiety level, or mental health status of the patient after the procedure; and
an electronic display configured to display the determined duration and/or the determined output.

20. The system of claim 19, wherein the imaging device includes a computed tomography (CT) imaging device configured to acquire at least one CT scan, and the controller is configured to:
execute one or more algorithms to determine, based on the at least one CT scan, the osteophyte volume, and
determine, based on the determined osteophyte volume, the duration of the medical procedure.
